(19) United States
(12) Patent Application Publication
(10) Pub. No.: US 2021/0238281 A1
(43) Pub. Date: Aug. 5, 2021

(54) HIGHLY SIALYLATED AUTOANTIBODIES AND USES THEREOF

(71) Applicants: INSTITUT NATIONAL DE LA SANTE ET DE LA RECHERCHE MEDICALE (INSERM), PARIS (FR); UNIVERSITÉ DE LILLE, LILLE (FR); LABORATOIRE FRANCAIS DU FRACTIONNEMENT ET DES BIOTECHNOLOGIES, LES ULIS (FR)

(72) Inventors: Céline MONNET, LAMBERSART (FR); Leonardus MARS, LAMBERSART (FR)

(21) Appl. No.: 17/049,093

(22) PCT Filed: Apr. 19, 2019
(86) PCT No.: PCT/EP2019/060240

§ 371 Date: Oct. 20, 2020

(30) Foreign Application Priority Data
Apr. 20, 2018 (FR) 18 53485

(51) Int. Cl. C07K 16/28; A61P 43/00

(52) U.S. Cl. CPC C07K 16/2803; A61K 2039/505; A61P 43/00

(57) ABSTRACT

The present invention relates to an isotype G antibody directed against native myelin oligodendrocytic glycoprotein (MOG), comprising:
an Fc fragment exhibiting high sialylation, and
an Fab fragment capable of binding to the autoantigen.

It also relates to a composition containing such an antibody, and to their uses in therapy.

The present invention relates to an isotype G antibody directed against an autoantigen, preferably against native myelin oligodendrocytic glycoprotein (MOG), comprising:
an Fc fragment exhibiting high sialylation, and
an Fab fragment capable of binding to the autoantigen.

It also relates to a composition containing such an antibody, and to its uses in therapy, in particular in the prevention and/or treatment of multiple sclerosis.

Autoimmune diseases occur when the immune response mistakenly targets the natural constituents of tissues and organs. The resulting inflammatory response interferes with the natural function of organs causing severe tissue damage, resulting in disease manifestations. Activation of T and B lymphocytes is common to all autoimmune diseases, and leads to deleterious cellular and humoral inflammatory responses. Due to the presence of T lymphocyte receptors and B lymphocytes, these responses are antigen specific, which, in the case of autoimmunity, imposes targeted aggression on tissue-derived autoantigens. This is because the antibodies and T lymphocytes isolated from the lesions readily react to autoantigens present in the inflamed tissue.

The treatment of organ-specific autoimmune diseases is currently based on palliative approaches that aim to deplete immune cells, block their migration to tissue damage, neutralize effector cytokines, or even the administration of intravenous immunoglobulins (IVIG). However, even if these treatments are effective, the natural course of the disease is restored once the treatment is finished.

Future therapies aimed at curing immune-mediated inflammatory diseases must increase their effectiveness to persist beyond the duration of treatment. In the case of organ-specific autoimmune diseases, this involves re-educating the immune system to restore immune tolerance.

One organ-specific autoimmune disease is multiple sclerosis (MS).

MS is a disease of the central nervous system (CNS). The CNS is made up of the brain and the spinal cord. At the microscopic level, the central nervous system is mainly composed of astrocytes, oligodendrocytes responsible for myelination, and neurons, each of which is made up of a cell body and an extension (axon), surrounded by a myelin sheath.

This myelin sheath serves to insulate and protect nerve fibers, and also plays a role in the speed of propagation of the nerve impulse carrying information along neurons.

MS is characterized by focal lesions in the white matter both in the brain and in the spinal cord. Pathological markers of the disease include demyelination, apoptosis of oligodendrocytes, axon scarring and finally neuronal loss. This tissue damage is caused by inflammation, as shown by the infiltration of lymphocytes and myeloid cells into the lesions. This pathophysiology causes difficulty in conduction of nerve impulses within axons, which causes motor, sensory and cognitive disturbances. In the more or less long term, these disorders can progress to an irreversible handicap.

Most commonly, MS begins with a recurrence-remission phase, during which periods of active clinical deficits are followed by prolonged periods of remission. Within the lesions, inflammation disappears and repair mechanisms (remyelination) allow the patient to regain proper nerve conduction. But unfortunately, in some advanced forms of MS or during severe inflammatory attacks, the remyelination mechanisms are overwhelmed, and irreversible nerve impulse conduction disturbances set in with corresponding neurological signs. Clinically, these patients progress to a secondary progressive course characterized by a gradual progression.

MS is considered to be an autoimmune disease. In MS, the immune system attacks antigenic targets in the CNS, including myelin. All the components of the immune response are involved: lymphocytes, myeloid cells, but also cytokines synthesized and released by immune cells which sometimes promote the attack and sometimes moderate it. The immune response in MS is not static, it is composite and evolves over time, both in terms of antigenic specificity and in pathogenic mechanisms. The DMARDs used today act either directly on lymphocytes, or by depleting them, or by inhibiting their migration to the CNS, to limit the extent of the inflammatory attack.

However, while current treatments reduce relapses and improve patients' quality of life, they are insufficiently effective in controlling disease progression.

There is therefore a need for an effective treatment of MS, which is in particular capable of slowing down and/or reducing the progression of the disease.

More generally, there is a need for an effective treatment of autoimmune diseases, and in particular autoimmune diseases specific to an organ.

The present invention addresses this problem.

It relates to an isotype G antibody directed against an autoantigen, comprising:
an Fc fragment exhibiting high sialylation, and
an Fab fragment capable of binding to the autoantigen.

More preferably, it relates to an isotype G antibody directed against the native myelin oligodendrocytic glycoprotein (MOG), comprising:
an Fc fragment exhibiting high sialylation, and
an Fab fragment capable of binding to native MOG.

In fact, as demonstrated in examples, the inventors have identified a specific anti-MOG IgG antibody capable of slowing down and/or reducing the progression of the disease. This antibody is derived from the pathogenic clone 8-18C5 (commercially available under the reference MAB5680 by Merck Millipore); it is capable of binding to the native human or murine MOG protein, but not to the linear fragment MOG₃₅₋₅₅.

In addition, this antibody has been modified compared to the pathogenic clone 8-18C5, in particular because it comprises a highly sialylated Fc fragment. More precisely, its Fc comprises a point deletion of glutamic acid in position 294 (the numbering being that of the EU index or equivalent in Kabat), which gives it an increased sialylation compared to the Fc which does not present this deletion.

This deletion confers in particular on the variant reduced binding affinities to FcγRIII and FcγRIIB, while the binding to FcRn is not affected; and anti-inflammatory properties. This antibody attenuates the severity of the disease in a mouse model of experimental autoimmune encephalomyelitis (EAE).

The definitions used in this application are as follows:

By “Fc fragment” or “Fc region” is meant the constant region of a full-length immunoglobulin (antibody) excluding the first constant region domain of immunoglobulin (i.e. CH1-CL). Thus the Fc fragment refers to a homodimer, each monomer comprising the last two constant domains of IgG (i.e. CH2 and CH3), and the N-terminal flexible hinge region of these domains. The Fc fragment of the antibody according to the invention is preferably a human Fc fragment and may be chosen from the Fc fragments of IgG1, IgG2, IgG3 and IgG4. Preferably, in the present invention, an Fc fragment of an IgG1 is used, which consists of the N-terminal flexible hinge and the CH2-CH3 domains, i.e. the portion from the amino acid C226 to the C-terminus, the numbering being indicated according to the EU index or equivalent in Kabat. Preferably, an Fc fragment of a human IgG1 (i.e. amino acids 226 to 447 according to the EU index or equivalent in Kabat) is used. In this case, the lower hinge refers to positions 226 to 230, the CH2 domain refers to positions 231 to 340 and the CH3 domain refers to positions 341-447 according to the EU index or equivalent in Kabat. The Fc fragment used according to the invention may also comprise a part of the upper hinge region, upstream of position 226. In this case, preferably, an Fc fragment of a human IgG1 comprising a part of the region is used. located between positions 216 to 226 (according to the EU index). In this case, the Fc fragment of a human IgG1 refers to the portion from amino acid 216, 217, 218, 219, 220, 221, 222, 223, 224 or 225 to the C terminal end.

Preferably, the Fc fragment of the antibody according to the invention is the Fc fragment of an IgG1.

The Fc fragment of the antibody according to the invention is preferably human.

In the present application, the numbering of the residues of the Fc fragment is that of the EU index or equivalent in Kabat (Sequences of Proteins of Immunological Interest, 5th Ed. Public Health Service, National Institutes of Health, Bethesda, Md. (1991)). This numbering is only suitable for human Fc fragments.

The equivalent of this numbering for murine (i.e. mouse or rat) Fc fragments is described in Zauner et al, Molecular & Cellular Proteomics 12.4, 2013, by the American Society for Biochemistry and Molecular Biology. This article describes in particular in FIG. 2 the differences in glycosylation between human and murine Fc.

By “amino acid mutation” is meant herein a change in the amino acid sequence of a polypeptide. A mutation is chosen in particular from a substitution, an insertion and a deletion.

By “substitution” is meant the replacement of one or more amino acids, at a particular position in a parent polypeptide sequence, with the same number of other amino acids. Preferably, the substitution is punctual, i.e. it concerns only a single amino acid. For example, the N434S substitution refers to a variant of a parent polypeptide, in which the asparagine at position 434 of the Fc fragment according to the EU index or equivalent in Kabat is replaced by serine.

By “insertion” is meant the addition of at least one amino acid at a particular position in a parent polypeptide sequence. For example, the insertion G>235-236 denotes an insertion of glycine between positions 235 and 236.

By “deletion” is meant the removal of at least one amino acid at a particular position in a parent polypeptide sequence. For example, E294del denotes the removal of glutamic acid at position 294.

By “parent polypeptide” and “parent antibody” are meant, respectively, a polypeptide or an unmodified antibody which is then modified to generate a variant. Said parent polypeptide or antibody may be of natural origin, a variant of a naturally occurring polypeptide or antibody, a modified version of a natural polypeptide or antibody or a synthetic polypeptide or antibody. Preferably, the parent polypeptide or antibody comprises an Fc fragment chosen from wild type Fc fragments, their fragments and their mutants. Therefore, the parent polypeptide or antibody may optionally include pre-existing amino acid modifications in the Fc fragment compared to wild type Fc fragments. Thus preferably, the Fc fragment of the parent polypeptide or antibody already comprises at least one additional mutation (i.e. pre-existing modification), preferably chosen from P230S, T256N, V259I, N315D, A330V, N361 D, A378V, S383N, M428L, N434Y.

Preferably, the Fc fragment of the parent polypeptide or antibody is chosen from the sequences SEQ ID NO: 1, 2, 3, 4 and 5. Preferably, the Fc fragment of the parent polypeptide or antibody has the sequence SEQ ID NO: 1.

The sequences shown in SEQ ID NO: 1, 2, 3, 4 and 5 are free from an N-terminal hinge region.

The sequences represented in SEQ ID NO: 6, 7, 8, 9 and 10 correspond respectively to the sequences represented in SEQ ID NO: 1, 2, 3, 4 and 5 with their N-terminal hinge regions. Also, in a particular embodiment, the Fc fragment of the parent polypeptide or antibody is chosen from the sequences SEQ ID NO: 6, 7, 8, 9 and 10.

Preferably, the Fc fragment of the parent polypeptide or antibody has a sequence corresponding to positions 1-232, 2-232, 3-232, 4-232, 5-232, 6-232, 7-232, 8-232, 9-232, 10-232 or 11-232 of the sequence SEQ ID NO: 6.

By “variant” is meant a polypeptide sequence which is different from the sequence of the parent polypeptide by at least one amino acid modification.

Preferably, the sequence of the variant has at least 80% identity with the sequence of the parent polypeptide, and more preferably at least 81%, 82%, 83%, 84%, 85%, 86%, 87%, 88%, 89%, 90%, 91%, 92%, 93%, 94%, 95%, 96%, 97%, 98%, 99% or 99.5% identity.

Throughout the present application, the expression “percentage identity” between two amino acid sequences within the meaning of the present invention is intended to denote a percentage of identical amino acid residues between the two sequences to be compared, obtained after the best alignment, this percentage being purely statistical and the differences between the two sequences being distributed at random and over their entire length. By “best alignment” or “optimal alignment” is meant the alignment for which the percentage identity determined as below is the highest. Sequence comparisons between two amino acid sequences are traditionally carried out by comparing these sequences after having optimally aligned them, said comparison being carried out by segment or by “comparison window” to identify and compare the local regions of sequence similarity. The optimal alignment of the sequences for the comparison can be achieved, besides manually, by means of the local homology algorithm of Smith and Waterman (1981, J. Mol Evol., 18: 38-46), by means of the ‘local homology algorithm of Neddleman and Wunsch (1970), by means of the similarity search method of Pearson and Lipman (1988, PNAS, 85: 2444-2448), by means of computer software using these algorithms (GAP, BESTFIT, BLAST P, BLAST N, FASTA, and TFASTA in the Wisconsin Genetics Software Package, Genetics Computer Group, 575 Science Dr., Madison, Wis.).

More preferably, the antibody according to the invention is chosen from IgG1, IgG2, IgG3 and IgG4, preferably is an IgG1.

The antibody according to the invention may be chimeric, humanized or human.

The term “chimeric” antibody is intended to denote an antibody which contains a natural variable region (light chain and heavy chain) derived from an antibody of a given species in association with the constant regions of light chain and heavy chain of an antibody of a species heterologous to said given species. Advantageously, if the antibody is chimeric, it comprises human constant regions. Starting from a non-human antibody (in particular murine), a chimeric antibody may be prepared using genetic recombination techniques well known to those skilled in the art. For example, the chimeric antibody could be produced by cloning for the heavy chain and the light chain a recombinant DNA comprising a promoter and a sequence encoding the variable region of the non-human antibody, and a sequence encoding the constant region of a human antibody. For the methods of preparing chimeric antibodies, reference may be made, for example, to the document Verhoeyn et al (Verhoeyn et al. BioEssays, 8: 74, 1988).

The term “humanized” antibody is understood to denote an antibody which contains complementarity determining regions (CDRs) derived from an antibody of non-human origin, the other parts of the antibody molecule being derived from one (or more) human antibodies. In addition, some of the residues of the framework regions (or “frameworks” or “FR”) may be modified to retain the binding affinity (Jones et al. Nature, 321: 522-525, 1986; Verhoeyen et al. 1988; Riechmann et al. Nature, 332: 323-327, 1988). The humanized antibodies may be prepared by techniques known to those skilled in the art, such as the “CDR grafting”, “resurfacing”, “Human string content”, “FR libraries”, “Guided selection”, “FR shuffling” and “Humaneering”, as summarized in the review by Almagro et al (Almagro et al. Frontiers in Bioscience 1 3, 1619-1633, Jan. 1, 2008).

By “human” antibody is meant an antibody the entire sequence of which is of human origin, that is to say the coding sequences of which have been produced by recombination of human genes coding for the antibodies. Indeed, it is now possible to produce transgenic animals (e.g. mice) which are capable, upon immunization, of producing a complete repertoire of human antibodies in the absence of endogenous production of immunoglobulin (see Jakobovits et al, Proc Natl Acad Sci USA 90: 2551 (1993); Jakobovits et al, Nature 362: 255-258 (1993); Bruggermann et al, Year in Immuno, 7:33 (1993); Duchosal et al. Nature 355: 258 (1992); U.S. Pat. Nos. 5,591,669; 5,598,369; 5,545,806; 5,545,807; U.S. Pat. No. 6,150,584). Human antibodies may also be obtained from phage display libraries (Hoogenboom et al, J. Mol. Biol, 227: 381 (1991); Marks et al, J. Mol. Biol, 222: 581-5597 (1991); Vaughan et al. Nature Biotech 14: 309 (1996)).

The antibody according to the invention is directed against an autoantigen.

By “autoantigen” is meant an antigen which, although being a constituent of normal tissue, is the target of a humoral or cellular immune response, as in the case of an autoimmune disease (see definition in Miller-Keane Encyclopedia).

Preferably, the antibody according to the invention is directed against an autoantigen chosen from the oligodendrocytic glycoprotein of native myelin (MOG), the catalytic 2 subunit of glucose-6 phosphatase (IGRP, encoded by the G6PC2 gene; Q9NQR9 in Uniprot), type 2 collagen and aquaporin-4 (P55087 in Uniprot).

These autoantigens are particularly relevant for the prevention and/or treatment of an autoimmune disease chosen from:
demyelinating diseases involving anti-MOG antibodies, such as multiple sclerosis;
Devic's neuromyelitis optic (NMO/NMOSD), by targeting aquaporin-4 (AQP-4) and/or MOG;
type 1 diabetes, by targeting the catalytic 2 subunit of glucose-6 phosphatase (IGRP). The IGRP is specific to the islets of Langerhans; and
rheumatoid arthritis, by targeting type 2 collagen.

The myelin oligodendrocytic glycoprotein (MOG) is one of several antigens in myelin and neurons to which immune reactivity is detected in MS. This glycoprotein is a minor component of the myelin sheath that isolates the axons of the CNS.

The sequence of this native human protein may be found in Uniprot with the accession number Q16653. The mature (native) human protein contains 218 amino acids (i.e. after cleavage of the signal peptide of 29 amino acids).

Similarly, the native mouse MOG sequence is accessible in Uniprot with accession number Q61885. The mature (native) mouse protein contains 218 amino acids (i.e. after cleavage of the 28 amino acid signal peptide). The native human and mouse MOG proteins are 89% identical.

Preferably, the invention relates to an isotype G antibody directed against native MOG, comprising:
an Fc fragment exhibiting high sialylation, and
an Fab fragment capable of binding to native MOG.

In particular, as detailed in Breithaupt et al, PNAS, Aug. 5, 2003, vol. 100, no. 16, the native MOG epitope consists of three loops located on the distal side of the MOG membrane, and in particular at the level residues 101-108 of sequence SEQ ID NO: 26 (R101DHSYQEE108, corresponding to residues 101-108 on the 218 of mature human MOG); these residues contain a loop which forms the upper edge of the putative ligand binding site.

Preferably, the invention relates to an isotype G antibody directed against native MOG, comprising:
an Fc fragment exhibiting high sialylation, and
an Fab fragment capable of binding to native MOG, in particular to residues 101-108 of sequence SEQ ID NO: 26.

Preferably, the antibody according to the invention is directed against native MOG. Preferably, it comprises the 6 CDRs of the murine antibody 8-18C5. Preferably, it includes the following 6 CDRs:
H-CDR1: SEQ ID NO: 11,
H-CDR2: SEQ ID NO: 12,
H-CDR3: SEQ ID NO: 13,
L-CDR1: SEQ ID NO: 14,
L-CDR2: GAS, and
L-CDR3: SEQ ID NO: 15.

According to a particular embodiment, the antibody directed against native MOG according to the invention is chimeric and comprises as VH the sequence SEQ ID NO: 16, and as VL the sequence SEQ ID NO: 17. According to a particular embodiment, the antibody according to the invention is chimeric, and comprises as heavy chain the sequence SEQ ID NO: 24 with the deletion of glutamic acid in position 294 in the numbering of the index EU or equivalent in Kabat, and as light chain the sequence SEQ ID NO: 25.

The present application also describes a murine antibody directed against native MOG; typically, it comprises as heavy chain the sequence SEQ ID NO: 19, this sequence comprising the deletion of glutamic acid at position 171, and as light chain the sequence SEQ ID NO: 20. Position 171 on murine Fc corresponds to position 294 on human Fc with index numbering EU or equivalent in Kabat.

Advantageously, the variable region of each of the light chains of the antibody directed against native MOG according to the invention is encoded by a sequence having at least 80%, preferably at least 85%, preferably at least 90%, of preferably at least 95%, preferably at least 99%, identity with the murine sequence SEQ ID NO: 17, and the variable region of each of the heavy chains of the antibody directed against native MOG according to the invention is encoded by a sequence having at least 80%, preferably at least 85%, preferably at least 90%, preferably at least 95%, preferably at least 99%, identity with the murine nucleic acid sequence SEQ ID NO: 16.

The antibodies of the invention are also understood to mean any antibody directed against native MOG possessing the CDR (Complementary Determining Region) regions of the 8-18C5 antibody, associated with FR regions (framework, highly conserved regions of variable regions, called also “frame”). Such antibodies have very comparable, preferably identical, affinity and specificity to the murine 8-18C5 antibody.

Preferably, as indicated above, the antibody directed against native MOG according to the invention comprises the 6 CDRs of the murine antibody 8-18C5. Preferably, it includes the following 6 CDRs:
H-CDR1: SEQ ID NO: 11,
H-CDR2: SEQ ID NO: 12,
H-CDR3: SEQ ID NO: 13,
L-CDR1: SEQ ID NO: 14,
L-CDR2: GAS, and
L-CDR3: SEQ ID NO: 15.

Advantageously, the FR regions of the VL region of the antibody directed against native MOG according to the invention is encoded by a sequence having at least 80%, preferably at least 85%, preferably at least 90%, preferably at least 95%, preferably at least 99%, identity with the FR regions of the murine sequence SEQ ID NO: 17, and the FR regions of the VH region of the antibody directed against native MOG according to the invention is encoded by a sequence having at least 80%, preferably at least 85%, preferably at least 90%, preferably at least 95%, preferably at least 99%, identity with the FR regions of the murine sequence SEQ ID NO: 16.

Advantageously, the antibody directed against native MOG according to the invention comprises, as Fc region, a human Fc region, preferably chosen from SEQ ID NO: 1 to 10, preferably the Fc region encoded by SEQ ID NO: 1, and comprising the deletion of glutamic acid at position 294 in the numbering of the index EU or equivalent in Kabat. The isotype G antibody directed against an autoantigen, and in particular directed against the oligodendrocytic glycoprotein of native myelin (MOG), according to the invention, may be obtained by selection on a phage bank, as in particular described in Nixon et al., Drugs derived from phage display, From candidate identification to practice, mAbs 6:1, 73-85; January/February 2014.

The present invention also relates to an antibody composition of isotype G as mentioned above, which comprises Fc fragments exhibiting high sialylation. This high sialylation to Fc is typically increased or improved over that of a parent antibody composition.

By “increased sialylation” or “improved sialylation” is meant that the sialylation of the Fc of the antibody composition obtained is increased by at least 10%, preferably at least 15%, preferably at least 20%, preferably at least 25%, preferably at least 30%, preferably at least 35%, preferably at least 40%, preferably at least 45%, preferably at least 50%, preferably at least 55%, of preferably at least 60%, preferably at least 65%, preferably at least 70%, preferably at least 75%, preferably at least 80%, preferably at least 85%, preferably at least 90%, preferably at least 95%, based on the sialylation of the Fc of said parent antibody composition.

Sialylation of a protein is a well-known glycosylation mechanism (see in particular Essentials of Glycobiology, 2nd edition, Varki et al, 2009). It corresponds to an addition, by covalent bond, of at least one sialic acid (i.e. N-acetylneuraminic acid and its derivatives, such as N-glycosylneuraminic acid, N-acetylglycosylneuraminic acid) in the glycosylated chain of the protein.

Preferably, the sialylation on the Fc fragment is obtained by mutation of the latter.

Thus, preferably, the Fc fragment, in particular human, is modified compared to that of a parent antibody and comprises at least one amino acid mutation chosen from amino acids in position 240 to 243, 258 to 267 and 290 to 305 of said Fc fragment, the numbering being that of the index EU or equivalent in Kabat.

Preferably, the mutation is carried out on at least one amino acid of the Fc fragment located at position 240, 241, 242, 243, 258, 259, 260, 261, 262, 263, 264, 265, 266, 267, 290, 291, 292, 293, 294, 295, 296, 297, 298, 299, 300, 301, 302, 303, 304 or 305, the numbering being that of the index EU or equivalent in Kabat.

Preferably, the mutation is chosen from V262del, V263F, V263K, V263W, V264K, V264P, D265A, D265E, D265G, D265L, D265S, D265V, V266A, V266P, V266S, V266T, S267N, S267P, S267R, S267W, P2910, P291V, P291Y, P291W, R292A, R292del, R292T, R292V, R292Y, E293del, E293F, E293P, E293W, E293Y, E294del, E294D, E294N, E294W, E294F, E293del/E294del, Q295D, Q295del, Q295F, Q295G, Q295K, Q295N, Q295R, Q295W, Y296A, Y296C, Y296del, Y296E, Y296G, Y296Q, Y296R, Y296V, S298del, S298E, S298F, S298G, S298L, S298M, S298N, S298P, S298R, S298T, S298W, S298Y, Y300D, Y300del, Y300G, Y300N, Y300P, Y300R, Y300S, R301A, R301F, R301G, R301H, R3011, R301K, R3010, R301V, R301W, R301Y, V302del, V302A, V302F, V302G, V302P, V303A, V303C, V303P, V303L, V303S, V303Y, S304C, S304M, S304Q, S304T, V305F and V305L, the numbering being that of the index EU or equivalent in Kabat.

More preferably, the Fc fragment of the antibody according to the invention is modified relative to that of a parent antibody and comprises at least the E294del mutation, the numbering being that of the EU index or equivalent in Kabat.

Preferably, the Fc fragment of the antibody according to the invention, in particular human, is modified relative to that of a parent antibody and consists of the E294del mutation, the numbering being that of the EU index or equivalent in Kabat.

According to the invention, when the Fc fragment of the antibody according to the invention is a mouse Fc, it is modified compared to that of a parent antibody, in particular of sequence SEQ ID NO: 18, and consists of the mutation E171del.

Preferably, the Fc fragment of the antibody according to the invention, in particular human, is modified relative to that of a parent antibody and consists of the Y300del mutation, the numbering being that of the EU index or equivalent in Kabat. Preferably, such an antibody is produced in HEK cells.

Preferably, the antibody according to the invention exhibits at least one effector activity mediated by said reduced Fc fragment relative to the effector activity of the parent antibody.

By “effector activity mediated by the Fc fragment” is meant in particular the cellular cytotoxicity dependent on the antibodies (ADCC or Antibody-Dependent Cell-mediated Cytotoxicity), the complement-dependent cytotoxicity (CDC or Complement Dependent Cytotoxicity), the cellular phagocytosis dependent on the antibodies. antibody (ADCP), endocytosis activity or the secretion of cytokines. Preferably, the effector activity mediated by the Fc fragment considered in the invention is selected from antibody-dependent cellular cytotoxicity (ADCC), complement-dependent cytotoxicity (CDC) and antibody-dependent cellular phagocytosis (ADCP) and secretion of cytokines.

By “reduced” effector activity is meant a reduced or abolished effector activity. Thus, the antibody according to the invention may exhibit at least one effector activity mediated by the abolished Fc fragment. Preferably, the antibody according to the invention exhibits an effector activity mediated by the Fc region which is reduced, relative to that of the parent antibody, of at least 10%, preferably of at least 20%, 30%, 40%, 50%, 60%, 70%, 80%, or 90%.

Preferably, the antibody according to the invention is devoid of any effector activity mediated by said Fc fragment.

According to another aspect, the antibody according to the invention exhibits an affinity mediated by the Fc fragment, reduced relative to the affinity of the parent antibody, for at least one of the receptors of the Fc region (FcR).

By “receptor of the Fc region” or “FcR” is meant in particular the C1q and the Fcγ receptors (FcγR). The “FcγReceptors” or “FcγR” refer to the IgG receptors, called CD64 (FcγRI), CD32 (FcγRII), and CD16 (FcγRIII), in particular to the five expressed receptors FcγRIa, FcγRIIa, FcγRIIb, FcγRIIIa and FcγRIIIb. All are receptor activators of effector cells, except for human FcγRIIb which is a receptor which inhibits the activation of immune cells (Muta T et al., Nature, 1994, 368: 70-73).

The C1q complement is involved in CDC activity.

The FcgRIIIa (CD16a) receptor is involved in ADCC; it presents a V/F polymorphism at position 158.

The FcgRIIa (CD32a) receptor is involved in platelet activation and phagocytosis; it shows an H/R polymorphism at position 131.

Finally, the FcgRIIb (CD32b) receptor is involved in the inhibition of cellular activity.

By “decreased” affinity is meant a decreased or abolished affinity. Preferably, the affinity is reduced, relative to that of the parent antibody comprising the Fc fragment, by at least 10%, preferably by at least 20%, 30%, 40%, 50%, 60%, 70%, 80%, or 90%.

Preferably, the antibody according to the invention exhibits an affinity mediated by said Fc fragment reduced relative to the affinity of the parent antibody, for at least one of the receptors of the Fc region (FcR) chosen from the complement C1q and the FcgRIIIa (CD16a), FcgRIIa (CD32a) and FcgRIIb (CD32b) receptors. Preferably, the antibody according to the invention exhibits an affinity mediated by said Fc fragment which is reduced relative to the affinity of the parent antibody, for the two receptors C1q and CD16a.

The affinity of an antibody comprising an Fc fragment for an FcR can be assessed by methods well known in the prior art. For example, those skilled in the art can determine the affinity (Kd) using, for example, surface plasmon resonance (SPR), or Octet® technology (BLI “Bio-Layer Interferometry” technology, Pall). Alternatively, one skilled in the art can perform an appropriate ELISA test. An appropriate ELISA assay allows comparison of the binding strengths of the parent Fc and the mutated Fc. The detected signals specific for the mutated Fc and the parent Fc are compared. The binding affinity may be determined either by evaluating whole antibodies or by evaluating the Fc regions isolated from them.

Preferably, the IgG-type antibody according to the invention is directed against native MOG and comprises:
the following 6 CDRs:
H-CDR1: SEQ ID NO: 11,
H-CDR2: SEQ ID NO: 12,
H-CDR3: SEQ ID NO: 13,
L-CDR1: SEQ ID NO: 14,
L-CDR2: GAS, and
L-CDR3: SEQ ID NO: 15; and
a human Fc fragment modified relative to that of a parent antibody, comprising at least one amino acid mutation chosen from amino acids in position 240 to 243, 258 to 267 and 290 to 305 of said Fc fragment, the numbering being that of the EU index or equivalent in Kabat; preferably a human Fc fragment modified from that of a parent antibody, comprising at least the E294del mutation (or at least the Y300del mutation), the numbering being that of the EU index or equivalent in Kabat.

Preferably, the IgG-type antibody according to the invention is directed against native MOG and comprises:
the following 6 CDRs:
H-CDR1: SEQ ID NO: 11,
H-CDR2: SEQ ID NO: 12,
H-CDR3: SEQ ID NO: 13,
L-CDR1: SEQ ID NO: 14,
L-CDR2: GAS, and
L-CDR3: SEQ ID NO: 15; and
a mouse Fc fragment modified from that of a parent antibody, comprising at least the E171del mutation (which corresponds to E294del on the human Fc fragment with the numbering of the EU index or equivalent in Kabat). Preferably, the mouse Fc fragment has the sequence SEQ ID NO: 18 and comprises the E171del mutation.

In a preferred embodiment, the IgG-type antibody as defined above is directed against native MOG and comprises:
a variable domain of the heavy chain comprising or consisting of a sequence selected from the group consisting of the sequence SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77 SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105 and SEQ ID NO: 107, and/or
a variable domain of the light chain comprising or consisting of a sequence selected from the group consisting of the sequence SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44 SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 6, OSEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106 and SEQ ID NO: 108.

In a preferred embodiment, the IgG-type antibody as defined above is directed against native MOG and comprises a heavy chain variable domain and a light chain variable domain of sequences:
SEQ ID NO: 29 and SEQ ID NO: 30,
SEQ ID NO: 31 and SEQ ID NO: 32,
SEQ ID NO: 33 and SEQ ID NO: 34,
SEQ ID NO: 35 and SEQ ID NO: 36,
SEQ ID NO: 37 and SEQ ID NO: 38,
SEQ ID NO: 39 and SEQ ID NO: 40,
SEQ ID NO: 41 and SEQ ID NO: 42,
SEQ ID NO: 43 and SEQ ID NO: 44,
SEQ ID NO: 45 and SEQ ID NO: 46,
SEQ ID NO: 47 and SEQ ID NO: 48,
SEQ ID NO: 49 and SEQ ID NO: 50,
SEQ ID NO: 51 and SEQ ID NO: 52,
SEQ ID NO: 53 and SEQ ID NO: 54,
SEQ ID NO: 55 and SEQ ID NO: 56,
SEQ ID NO: 57 and SEQ ID NO: 58,
SEQ ID NO: 59 and SEQ ID NO: 60,
SEQ ID NO: 61 and SEQ ID NO: 62,
SEQ ID NO: 63 and SEQ ID NO: 64,
SEQ ID NO: 65 and SEQ ID NO: 66,
SEQ ID NO: 67 and SEQ ID NO: 68,
SEQ ID NO: 69 and SEQ ID NO: 70,
SEQ ID NO: 71 and SEQ ID NO: 72,
SEQ ID NO: 73 and SEQ ID NO: 74,
SEQ ID NO: 75 and SEQ ID NO: 76,
SEQ ID NO: 77 and SEQ ID NO: 78,
SEQ ID NO: 79 and SEQ ID NO: 80,
SEQ ID NO: 81 and SEQ ID NO: 82,
SEQ ID NO: 83 and SEQ ID NO: 84,
SEQ ID NO: 85 and SEQ ID NO: 86,
SEQ ID NO: 87 and SEQ ID NO: 88,
SEQ ID NO: 89 and SEQ ID NO: 90,
SEQ ID NO: 91 and SEQ ID NO: 92,
SEQ ID NO: 93 and SEQ ID NO: 94,
SEQ ID NO: 95 and SEQ ID NO: 96,
SEQ ID NO: 97 and SEQ ID NO: 98,
SEQ ID NO: 99 and SEQ ID NO: 100,
SEQ ID NO: 101 and SEQ ID NO: 102,
SEQ ID NO: 103 and SEQ ID NO: 104,
SEQ ID NO: 105 and SEQ ID NO: 106, or
SEQ ID NO: 107 and SEQ ID NO: 108.

The sequences described in the present application may be summarized as follows (for information, the glutamic acid of Fc in position 294 according to Kabat is indicated in bold underlined in the human sequences):

| SEQ ID | | |
|---|---|---|
| NO: | Definition | Sequence |
| 1 | Fc fragment of human | CPPCPAPELLGGPSVFLFPP |
| | IgG1 G1m1.17 (residues | KPKDTLMISRTPEVTCVVVD |
| | 226-447 according to the | VSHEDPEVKFNWYVDGVEVH |
| | EU index or equivalent in | NAKTKPREEQYNSTYRVVSV |
| | Kabat) without N-terminal | LTVLHQDWLNGKEYKCKVSN |
| | hinge region | KALPAPIEKTISKAKGQPRE |
| | | PQVYTLPPSRDELTKNQVSL |
| | | TCLVKGFYPSDIAVEWESNG |
| | | QPENNYKTTPPVLDSDGSFF |
| | | LYSKLTVDKSRWQQGNVFSC |
| | | SVMHEALHNHYTQKSLSLSPGK |
| 2 | Fc fragment of human | CPPCPAPPVAGPSVFLFPPK |
| | IgG2 without N-terminal | PKDTLMISRTPEVTCVVVDV |
| | hinge region | SHEDPEVQFNWYVDGVEVHN |
| | | AKTKPREEQFNSTFRVVSVL |
| | | TVVHQDWLNGKEYKCKVSNK |
| | | GLPAPIEKTISKTKGQPREP |
| | | QVYTLPPSREEMTKNQVSLT |
| | | CLVKGFYPSDIAVEWESNGQ |
| | | PENNYKTTPPMLDSDGSFFL |
| | | YSKLTVDKSRWQQGNVFSCS |
| | | VMHEALHNHYTQKSLSLSPGK |
| 3 | Fc fragment of human | CPRCPAPELLGGPSVFLFPP |
| | IgG3 without N-terminal | KPKDTLMISRTPEVTCVVVD |
| | hinge region | VSHEDPEVQFKWYVDGVEVH |
| | | NAKTKPREEQYNSTFRVVSV |
| | | LTVLHQDWLNGKEYKCKVSN |
| | | KALPAPIEKTISKTKGQPRE |
| | | PQVYTLPPSREEMTKNQVSL |
| | | TCLVKGFYPSDIAVEWESSG |
| | | QPENNYNTTPPMLDSDGSFF |
| | | LYSKLTVDKSRWQQGNIFSC |
| | | SVMHEALHNRFTQKSLSLSPGK |
| 4 | Fc fragment of human | CPSCPAPEFLGGPSVFLFPP |
| | IgG4 without N-terminal | KPKDTLMISRTPEVTCVVVD |
| | hinge region | VSQEDPEVQFNWYVDGVEVH |
| | | NAKTKPREEQFNSTYRVVSV |
| | | LTVLHQDWLNGKEYKCKVSN |
| | | KGLPSSIEKTISKAKGQPRE |
| | | PQVYTLPPSQEEMTKNQVSL |
| | | TCLVKGFYPSDIAVEWESNG |
| | | QPENNYKTTPPVLDSDGSFF |
| | | LYSRLTVDKSRWQEGNVFSC |
| | | SVMHEALHNHYTQKSLSLSLGK |
| 5 | Fc fragment of human | CPPCPAPELLGGPSVFLFPP |
| | IgG1 G1m3 without N- | KPKDTLMISRTPEVTCVVVD |
| | terminal hinge region | VSHEDPEVKFNWYVDGVEVH |
| | | NAKTKPREEQYNSTYRVVSV |
| | | LTVLHQDWLNGKEYKCKVSN |
| | | KALPAPIEKTISKAKGQPRE |
| | | PQVYTLPPSREEMTKNQVSL |
| | | TCLVKGFYPSDIAVEWESNG |
| | | QPENNYKTTPPVLDSDGSFF |
| | | LYSKLTVDKSRWQQGNVFSC |
| | | SVMHEALHNHYTQKSLSLSPGK |
| 6 | Fragment Fc Fc fragment | EPKSCDKTHTCPPCPAPELL |
| | of human IgG1 G1m1.17 | GGPSVFLFPPKPKDTLMISR |
| | (residues 226-447 | TPEVTCVVVDVSHEDPEVKF |
| | according to the EU index | NWYVDGVEVHNAKTKPREEQ |
| | or equivalent in Kabat) | YNSTYRVVSVLTVLHQDWLN |
| | with N-terminal hinge | GKEYKCKVSNKALPAPIEKT |
| | region | ISKAKGQPREPQVYTLPPSR |
| | | DELTKNQVSLTCLVKGFYPS |
| | | DIAVEWESNGQPENNYKTTP |
| | | PVLDSDGSFFLYSKLTVDKS |
| | | RWQQGNVFSCSVMHEALHNH |
| | | YTQKSLSLSPGK |
| 7 | Fc fragment of human | ERKCCVECPPCPAPPVAGPS |
| | IgG2 with N-terminal | VFLFPPKPKDTLMISRTPEV |
| | hinge region | TCVVVDVSHEDPEVQFNWYV |
| | | DGVEVHNAKTKPREEQFNST |
| | | FRVVSVLTVVHQDWLNGKEY |
| | | KCKVSNKGLPAPIEKTISKT |
| | | KGQPREPQVYTLPPSREEMT |
| | | KNQVSLTCLVKGFYPSDIAV |
| | | EWESNGQPENNYKTTPPMLD |
| | | SDGSFFLYSKLTVDKSRWQQ |
| | | GNVFSCSVMHEALHNHYTQK |
| | | SLSLSPGK |
| 8 | Fc fragment of human | ELKTPLGDTTHTCPRCPEPK |
| | IgG3 with N-terminal | SCDTPPPCPRCPEPKSCDTP |
| | hinge region | PPCPRCPEPKSCDTPPPCPR |
| | | CPAPELLGGPSVFLFPPKPK |
| | | DTLMISRTPEVTCVVVDVSH |
| | | EDPEVQFKWYVDGVEVHNAK |
| | | TKPREEQYNSTFRVVSVLTV |
| | | LHQDWLNGKEYKCKVSNKAL |
| | | PAPIEKTISKTKGQPREPQV |
| | | YTLPPSREEMTKNQVSLTCL |
| | | VKGFYPSDIAVEWESSGQPE |
| | | NNYNTTPPMLDSDGSFFLYS |
| | | KLTVDKSRWQQGNIFSCSVM |
| | | HEALHNRFTQKSLSLSPGK |
| 9 | Fc fragment of human | ESKYGPPCPSCPAPEFLGGP |
| | IgG4 with N-terminal | SVFLFPPKPKDTLMISRTPE |
| | hinge region | VTCVVVDVSQEDPEVQFNWY |
| | | VDGVEVHNAKTKPREEQFNS |
| | | TYRVVSVLTVLHQDWLNGKE |
| | | YKCKVSNKGLPSSIEKTISK |
| | | AKGQPREPQVYTLPPSQEEM |
| | | TKNQVSLTCLVKGFYPSDIA |
| | | VEWESNGQPENNYKTTPPVL |
| | | DSDGSFFLYSRLTVDKSRWQ |
| | | EGNVFSCSVMHEALHNHYTQ |
| | | KSLSLSLGK |
| 10 | Fc fragment of human | EPKSCDKTHTCPPCPAPELL |
| | IgG1 G1m3 with N- | GGPSVFLFPPKPKDTLMISR |
| | terminal hinge region | TPEVTCVVVDVSHEDPEVKF |
| | | NWYVDGVEVHNAKTKPREEQ |
| | | YNSTYRVVSVLTVLHQDWLN |
| | | GKEYKCKVSNKALPAPIEKT |
| | | ISKAKGQPREPQVYTLPPSR |
| | | EEMTKNQVSLTCLVKGFYPS |
| | | DIAVEWESNGQPENNYKTTP |
| | | PVLDSDGSFFLYSKLTVDKS |
| | | RWQQGNVFSCSVMHEALHNH |
| | | YTQKSLSLSPGK |
| 11 | H-CDR1 from murine | GYTFSSFW |
| | antibody 8-18C5 | |
| 12 | H-CDR2 from murine | ILPGRGRT |
| | antibody 8-18C5 | |
| 13 | H-CDR3 from murine | ATGNTMVNMPY |
| | antibody 8-18C5 | |
| 14 | L-CDR1 of murine | QSLLNSGNQKNY |
| | antibody 8-18C5 | |
| 15 | L-CDR3 of murine | QNDHSYPL |
| | antibody 8-18C5 | |
| 16 | VH of the recombinant | EVKLHESGAGLVKPGASVEISCKAT |
| | murine 8-18C5 antibody | GYTFSSFWIEWVKQRPGHGLEWIGE |
| | | ILPGRGRTNYNEKFKGKATFTAETSS |
| | | NTAYMQLSSLTSEDSAVYYCATGNT |
| | | MVNMPYWGQGTTVTVSS |
| 17 | VL of the recombinant | DIELTQSPSSLAVSAGEKVT |
| | murine 8-18C5 antibody | MSCKSSQSLLNSGNQKNYL |
| | | AWYQQKPGLPPKLLIYGAST |
| | | RESGVPDRFTGSGSGTDFTL |
| | | TISSVQAEDLAVYYCONDHSY |
| | | PLTFGAGTKLEIK |
| 18 | Constant region (CH1- | AKTTPPSVYPLAPGSAAQTN |
| | hinge-CH2-CH3) of | SMVTLGCLVKGYFPEPVTVT |
| | recombinant murine | WNSGSLSSGVHTFPAVLESD |
| | antibody 8-18C5 (Note | LYTLSSSVTVPSSPRPSETVT |
| | glutamic acid at position | CNVAHPASSTKVDKKIVPRDC |
| | 171, which corresponds to | GCKPCICTVPEVSSVFIFPPKP |
| | position 294 on human Fc | KDVLTITLTPKVTCVVVDISKD |
| | with the numbering of the | DPEVQFSWFVDDVEVHTAQTQ |
| | EU index or equivalent in | PREEQFNSTFRSVSELPIMHQD |
| | Kabat. It is indicated in | WLNGKEFKCRVNSAAFPAPIEK |
| | underlined bold) | TISKTKGRPKAPQVYTIPPPKEQ |
| | | MAKDKVSLTCMITDFFPEDITVE |
| | | WOWNGOPAENYKNTQPIMNTN |
| | | GSYFVYSKLNVQKSNWEAGNT |
| | | FTCSVLHEGLHNHHTEKSLSHSPGK |
| 19 | Recombinant murine 8- | EVKLHESGAGLVKPGASVEIS |
| | 18C5 heavy chain | CKATGYTFSSFWIEWVKQR |
| | (Glutamic acid is found | PGHGLEWIGEILPGRGRTN |
| | at position 171 of SEQ ID | YNEKFKGKATFTAETSSNT |
| | NO: 18, which | AYMQLSSLTSEDSAVYYCAT |
| | corresponds to position | GNTMVNMPYWGQGTTVTV |
| | 294 on human Fc with the | SSAKTTPPSVYPLAPGSAAQ |
| | numbering of the EU | TNSMVTLGCLVKGYFPEPVTVTW |
| | index or equivalent in | NSGSLSSGVHTFPAVLESDL |
| | Kabat. It is indicated in | YTLSSSVTVPSSPRPSETVT |
| | bold underlined italics) | CNVAHPASSTKVDKKIVPRD |
| | | CGCKPCICTVPEVSSVFIFPP |
| | | KPKDVLTITLTPKVTCVVVDISK |
| | | DDPEVQFSWFVDDVEVHTAQ |
| | | TQPRE [symbol] QFNSTFRSVSELPIMH |
| | | QDWLNGKEFKCRVNSAAFPA |
| | | PIEKTISKTKGRPKAPQVYTIP |
| | | PPKEQMAKDKVSLTCMITDFF |
| | | PEDITVEWQWNGQPAENYKNT |
| | | QPIMNTNGSYFVYSKLNVQKSN |
| | | WEAGNTFTCSVLHEGL |
| | | HNHHTEKSLSHSPGK |
| 20 | Recombinant murine 8- | DIELTQSPSSLAVSAGEKVTMS |
| | 18C5 antibody light chain | CKSSQSLLNSGNQKNYLAWYQQ |
| | | KPGLPPKLLIYGASTRESGVPDRF |
| | | TGSGSGTDFTLTISSVQAEDLAVYY |
| | | CQNDHSYPLTFGAGTKLEIKRADAAP |
| | | TVSIFPPSSEQLTSGGASVVCFLNNF |
| | | YPKDINVKWKIDGSERQNGVLNSWT |
| | | DQDSKDSTYSMSSTLTLTKDEYERH |
| | | NSYTCEATHKTSTSPIVKSFNRNEC |
| 21 | VH of murine hybridoma | QVQLQQSGAELMKPGAS |
| | 8-18C5 antibody | VEISCKATGYTFSSFWIEW |
| | | VKQRPGHGLEWIGEILPGR |
| | | GRTNYNEKFKGKATFTAET |
| | | SSNTAYMQLSSLTSEDSA |
| | | VYYCATGNTMVNMPYWGQ |
| | | GTTLTVSS |
| 22 | VL of murine hybridoma | DIVMTQSPSSLSVSAGEKVT |
| | 8-18C5 antibody | MSCKSSQSLLNSGNQKNYL |
| | | AWYQQKPGLPPKLLIYGAST |
| | | RESGVPDRFTGSGSGTDFTL |
| | | TISSVQAEDLAVYYCQNDHSY |
| | | PLTFGAGTKLELK |
| 23 | Constant region (CH1- | AKTTPPSVYPLAPGSAAQTNS |
| | hinge-CH2-CH3) of | MVTLGCLVKGYFPEPVTVTWN |
| | murine hybridoma 8-18C5 | SGSLSSGVHTFPAVLQSDLYTLS |
| | antibody | SSVTVPSSTWPSETVTCNVAHP |
| | | ASSTKVDKKIVPRDCGCKPCICT |
| | | VPEVSSVFIFPPKPKDVLTITLTPK |
| | | VTCVVVDISKDDPEVQFSWFV |
| | | DDVEVHTAQTQPREEQFNSTFRSVS |
| | | ELPIMHQDWLNGKEFKCRVNSAAF |
| | | PAPIEKTISKTKGRPKAPQVYTIPP |
| | | PKEQMAKDKVSLTCMITDFFPEDIT |
| | | VEWQWNGQPAENYKNTQPIMDTDGSY |
| | | FVYSKLNVQKSNWEAGNTFTCSVLHEG |
| | | LHNHHTEKSLSHSPGK |
| 24 | Chimeric 8-18C5 heavy | EVKLHESGAGLVKPGASVEISCKATGYTF |
| | chain (Glutamic acid is | SSFWIEWVKQRPGHGLEWIGEILPGRGRT |
| | found at position 294 on | NYNEKFKGKATFTAETSSNTAYMQLSSLT |
| | human Fc with the | SEDSAVYYCATGNTMVNMPYWGQGTTVT |
| | numbering of the EU | VSSASTKGPSVFPLAPSSKSTSGGTAALG |
| | index or equivalent in | CLVKDYFPEPVTVSWNSGALTSGVHTFPA |
| | Kabat. It is indicated in | VLQSSGLYSLSSVVTVPSSSLGTQTYICNV |
| | bold underlined italics) | NHKPSNTKVDKKVEPKSCDKTHTCPPCPA |
| | | PELLGGPSVFLFPPKPKDTLMISRTPEVTC |
| | | VVVDVSHEDPEVKFNWYVDGVEVHNAKT |
| | | KPRE [symbol] QYNSTYRVVSVLTVLHQDWLNGK |
| | | EYKCKVSNKALPAPIEKTISKAKGQPREPQ |
| | | VYTLPPSRDELTKNQVSLTCLVKGFYPSDI |
| | | AVEWESNGQPENNYKTTPPVLDSDGSFF |
| | | LYSKLTVDKSRWQQGNVFSCSVMHEALH |
| | | NHYTQKSLSLSPGK |
| 25 | Light chain chimeric 8- | DIELTQSPSSLAVSAGEKVTMSCKSSQSLL |
| | 18C5 antibody | NSGNQKNYLAWYQQKPGLPPKLLIYGAST |
| | | RESGVPDRFTGSGSGTDFTLTISSVQAEDL |
| | | AVYYCQNDHSYPLTFGAGTKLEIKRTVAAP |
| | | SVFIFPPSDEQLKSGTASVVCLLNNFYPRE |
| | | AKVQWKVDNALQSGNSQESVTEQDSKDS |
| | | TYSLSSTLTLSKADYEKHKVYACEVTHQGL |
| | | SSPVTKSFNRGEC |
| 26 | Native human MOG | RDHSYQEE |
| | epitope | |
| 29 | VH of anti-MOG MO4H- | MAGSLQVDQVQLVQSGTEVKKPGASVKVSCKVS |
| | 03 antibody | GYTLTELSMHWVRQAPGKGLEWMGGFDPEDGE |
| | | TIYAQKFQGRVTMTEDTSTDTAYMELSSLRSEDT |
| | | AVYYCATGATGAFDIWGQGTTVTVSS |
| 30 | VL of anti-MOG MO4H-03 | DIVMTQTPLSSPVTLGQPASISCRSSQSLVDSDG |
| | antibody | NTYLNWLQQRPGQPPRLLIYKISNRFSGVPDRFS |
| | | GSGAGTEFTLKISRVEAEDVGVYYCMQATQFPHT |
| | | FGQGTKLEIK |
| 31 | VH of anti-MOG MO4H- | MAGSLQVDEVQLVQSGAEVKKPVASVKVSCKAS |
| | 04 antibody | GYTFTSYGISWVRQAPGQGLEWMGWISAYNGNT |
| | | NYAQKLQGRVTMTTDTSTSTAYMELRSLRSDDTA |
| | | VYYCARNMGCSSTSCFVSWFDPWGQGTLVTVS |
| | | S |
| 32 | VL of anti-MOG MO4H-04 | QSALTQPPSASGSPGQSVTISCTGTHSDVGSFDS |
| | antibody | VSWYQQHPDKAPKLIIYDVNKRPAGVPHRFSGSK |
| | | SGNTASLTVSGLQSEDEADYYCNSYAGVDNFVF |
| | | GTGTKVTVL |
| 33 | VH of anti-MOG MO4H- | MAGSLQVDQMQLV.SGAEVKKPGESLKISCKGSG |
| | 37 antibody | YSFTSYWIGWVRQMPGKGLEWMGIIYPDDSDFR |
| | | YSPSFQGRVTILLDRSINTAYLQLSSLQASDTAMY |
| | | YCARREAVTAAPFDFWGQGTLVTVSS |
| 34 | VL of anti-MOG MO4H-37 | QSVLTQPPSASGAPGQRVSISCSGSSSNIGTNHV |
| | antibody | YWYQQFTGMAPKLIIDTNNQRPSGVPDRFSGSKS |
| | | GTSASLAISGLQSDDAADYYCAAWDDSLNGYGF |
| | | GSGTQLTVL |
| 35 | VH of anti-MOG MO4H- | MAGSLQVDEVQLLESGGGLVQPGGVPETLLCNL |
| | 38 antibody | WIHLQLLDALGPPSSREGAGVGLTYNSDGSSTTY |
| | | ADSVKDRFTISRDNSKNTLYLQMNSLRADDTAVY |
| | | YCAKEHRTGGDPGGLSWNFDLWGRGTLVTVSS |
| 36 | VL of anti-MOG MO4H-38 | QSVLTQPASVSGSPGQSITISCTGTSRDVGRYNY |
| | antibody | VSWYQQHPGKAPKLMIYEGSKRPSGVPDRFSGS |
| | | KSGNTASLSISGLQSEDEADYYCAAWDDTLNGEV |
| | | FGTGTKVTVL |
| 37 | VH of anti-MOG MO4H- | MAGSLQVDQVQLVESGGGLVQPGRSLRLSCAAS |
| | 40 antibody | GFTFDDYAMHWVRQAPGKGLEVSGISWNSGSIG |
| | | YADSVKGRFTISRDNAKNSLYLQMNSLRGEDTAV |
| | | YYCAKFPGGSIGYWGPGTLVTVSS |
| 38 | VL of anti-MOG MO4H-40 | DIVMTQSPSTLSASVGDRVTITCRASQGIRNDLG |
| | antibody | WYQQKPGKAPKLLIYAASSLQSGVPSRFSGSGS |
| | | GTDFTLTISSLQPEDFATYYCQQGRGTFGPGTKV |
| | | EIK |
| 39 | VH of anti-MOG MO4H- | MAGSLQVDQVQLVQSGAEVKKPGASVKVSCKAS |
| | 46 antibody | GYTFTSYAMHWVRQAPGQRLEWMGWINAGNGN |
| | | AKYSQKFQGRVTLTRDTSASTAYMKLSSLRSEDT |
| | | AVYYCARGAPTYRYFDLWGRGTLVTVSS |
| 40 | VL of anti-MOG MO4H-46 | QSVLTQPPSVSAAPGQKVTISCSGSSSNIGNNYV |
| | antibody | AWYQQLPGTAPKLLIYDNNKRPSGIPDRFSGSKS |
| | | GTSATLGITGLQTGDEADYYCGTSDSSLSAVVFG |
| | | GGTKLTVL |
| 41 | VL of anti-MOG MO3B-03 | MAGSLQVDQVQLQQSGPGLVRPSQTLSLTCAISG |
| | antibody | DSVSSSSAAWNWVRSPSRGLEWLGRTYYRSKW |
| | | YYDYAVSVKNRIAINPDTSKNQFSLHLNSVTPEDT |
| | | AVYYCATGWLRGHLDYWGQGTLVTVSS |
| 42 | VH of anti-MOG MO3B-03 | AIQMTQSPSSVSASVGDRVTITCRATQSISTYLNW |
| | antibody | YQQKVGRGPKLLVYAASRLQTGVPSRFSGSGSG |
| | | TDFTLTISSLQPEDSATYYCQQSYSAPPAFGGGT |
| | | KVEIK |
| 43 | VH of anti-MOG MO3F-02 | MAGSLQVDQVQLQQSGPGLVKPSQTLSLTCAISG |
| | antibody | DSVSSNSAAWNWIRKSPSRGLEWLGRTYYRSKW |
| | | YNDYAVSVKSRITINPDTSKNQFSLQLSSVTPEDT |
| | | AVYYCARASAGTFGYWGQGTLVTVSS |
| 44 | VL of anti-MOG MO3F-02 | DIVMTQSPSSLSASVGDRVTMTCRASQTINTYLN |
| | antibody | WYQQKPGKAPKLLIYAASTLQSGVPSRFSGSGSG |
| | | TDFSLTISSLQPEDFATYYCQHGYNNPPFTFGPG |
| | | TKVDIK |
| 45 | VH of anti-MOG MO4E-48 | MAGSLQVDQVQLQ.SGLGLVKPSQTLSLTCAISG |
| | antibody | DSVSSNSAAWNWIRQSPSRGLEWLGRTYYRSK |
| | | WINDYAVSVKSRITINPDTSKNQFSLQLNSVTPED |
| | | TAVYYCARAGGGSGLLDPWGQGTLVTVSS |
| 46 | VL of anti-MOG MO4E-48 | DVVMTQSPAVLSVTPGEKVTITCRASEGIGNYLY |
| | antibody | WYQQKPDQALKLLINYASQSISGVPSRFSGSGSG |
| | | TDFTFSISSLEAEDAAVYFCLQSYRLPLTFGGGTK |
| | | VEIK |
| 47 | VH of anti-MOG MO4B-42 | MAGSLQVDQVQLVQSGAEVKRPGESLKISCEGS |
| | antibody | GYSFTSSWIGWVRQMPGKGLECMGIIYPGDSDT |
| | | RYSPSFQGHVTISADKSISTAYLQWSSLRASDTA |
| | | MYYCARAYHSDYGFDFWGQGTLVTVSS |
| 48 | VL of anti-MOG MO4B-42 | EIVLTQPLSVSESPGKTVTISCTRSSGSIANNFVQ |
| | antibody | WYQRRPGSSPTTVIYENDQRPSGVPDRFSGSIDS |
| | | SSNSASLTITGLETQDEADYYCQSFNDDVGGGNS |
| | | GGGTK |
| 49 | VH of anti-MOG MO4B-43 | MAGSLQVDEVQLLESGGGLVPGGSLRLSCEVSG |
| | antibody | FSFSNHAMHWVRQAPGKALEHLSVLGSDGRSTY |
| | | YADSVKGRFTISRDISKTTVYLQMGSLRPGDMGV |
| | | YYCARGLYGDHWDASDLWGQGTMVTVSS |
| 50 | VL of anti-MOG MO4B-43 | EIVMTQSPATLSVSPAERVILSCRASQSVGNNVA |
| | antibody | WFQQKPGQAPRLLIHGASSRATGIPTRFSGSGSG |
| | | TELTLTISSLQSEDFAVYYCQQYGSAPITFGQGTR |
| | | LEIK |
| 51 | VH of anti-MOG MO3J-05 | MAGSLQVDEVQLVQSGAEVKKPGASVKVSCKAS |
| | antibody | GYTFTSYGISWVRQAPGQGLEWMGWISAYNGNT |
| | | NYAQKLQGRVTMTTDTSTSTAYMELRSLRSDDTA |
| | | VYYCARNMGCSSTSCFVSWFDPWGQGTLVTVS |
| | | S |
| 52 | VL of anti-MOG MO3J-05 | QPVLTQPPSASGTPGQRVTISCPGSSSNIGSNTV |
| | antibody | NWYQQLPGTAPKLLIYSNNQRPSGVPDRFSGSK |
| | | SGTSASLAISGLQSEDEADYYCAAWDDSLNGWV |
| | | FGGGTKLTVL |
| 53 | VH of anti-MOG MO3J-11 | MAGSLQVDQMQLVQSGAEVKKPGASVKVSCKAP |
| | antibody | GYTFTDYYIHWVRQAPGQGPEWMGWINPNSGG |
| | | TNYAQKF.GRVTMTRGTSISTAYMELSRLTSDDTA |
| | | VYYCARDQRRSSPYYLGYWDQGTLVTVSS |
| 54 | VL of anti-MOG MO3J-11 | QSVLTQPPSVSGAPGQRVTIPCTGSSSNIASYDV |
| | antibody | HWYQQLPGTAPKLLIYGNTNRPSGVPDRFSGSKS |
| | | GTSASLAITGLQAEDEADYYCQSYDSSLSGSVFG |
| | | GGTKLTVL |
| 55 | VH of anti-MOG MO3J-12 | MAGSLQVDEVQLLESGGGLVQPGGSLRLSCAAS |
| | antibody | GFTFSTYWMHWVRQAPGRGLVWVSRINTDGSST |
| | | DYADSVKGRFTISRDNAKNTLYLQMNSLRAEDTA |
| | | VYSCARGGQLVAAANDNWLDPWGQGTLVTVSS |
| 56 | VL of anti-MOG MO3J-12 | AIQLTQSPSSLSASAGDRVTITCRASQSINNYLNW |
| | antibody | YQQKPGKAPKVLIYGASNLQSGVPSRFSGSGSGT |
| | | DFTLTISSLQPEDFATYYCQQSYSTPRTFGQGTK |
| | | VEIK |
| 57 | VH of anti-MOG MO3J-19 | MAGSLQVDEVQLVQSGAEVKKPGASVKVSCKAS |
| | antibody | GYTFTSYGISWVRQAPGQGLEWMGWISAYNGNT |
| | | NYAQKLQGRVTMTTDTSTSTAYMELRSLRSDDTA |
| | | VYYCARNMGCSSTSCFVSWFDPWGQGTLVTVS |
| | | S |
| 58 | VL of anti-MOG MO3J-19 | QSVLTQPPSASGTPGQRVTISCSGSTSNIGSQIVN |
| | antibody | WYQQLPGTAPRLIIYNDNERPSGVSDRFSGSKSD |
| | | TSASLAISG LQSEDEADYYCAAWDDSLNGYVFGT |
| | | GTKVTVL |
| 59 | VH of anti-MOG MO3J-23 | MAGSLQVDQVQLVQSGAEVKPGASVKVSCKASG |
| | antibody | YTFTSYGISWVRQAPGQGLEWMGWISAYNGNTN |
| | | YAQKLQGRVTMTTDTSTSTAYMELRSLRSDDTAV |
| | | YYCARDPVGSLRPYYMDVWGEGTTVTVSS |
| 60 | VL of anti-MOG MO3J-23 | DIVMTQSPSTLSASVGDRVTITCRASQSISTWLAW |
| | antibody | YQQKPGKAPKLLIYAASSLQSGVPSRFSGSGSGT |
| | | DFTLTISSLQPEDFATYYCQQSYSTPRTFGQGTK |
| | | VEIK |
| 61 | VH of anti-MOG MO3J-26 | MAGSLQVDQVQLVQSGAEVKKPGSSVKVSCKAS |
| | antibody | GGTFSTYTLSWVRQAPGKGLEWMGGFDPEDGE |
| | | TIYAQKFQGRVTMTEDTSTDTAYLELSSLRSDDTA |
| | | VYYCAADEFWGPGTLVTVSS |
| 62 | VL of anti-MOG MO3J-26 | QSVLTQPPSVSAAPGQTVTISCSGSSSNIGNNYV |
| | antibody | SWYQQLPGTAPKLLIYDNNKRPSGIPDRFSGSKS |
| | | GTSATLGITGLQTGDEADYYCGTWDSSLSAVVFG |
| | | GGTKLTVL |
| 63 | VH of anti-MOG MO3J-37 | MAGSLQVDEVQLVESGGGLVQPGRSLRLSCAAS |
| | antibody | GFTFDDYAMHWVRQAPGKGLEWVSGISWNSGSI |
| | | GYADSVKGRFTISRDNAKNSLYLQMNSLRAEDTA |
| | | LYYCAKDMRAVAGTEGAFDIWGQGTMVTVSS |
| 64 | VL of anti-MOG MO3J-37 | QSVVTQPPSMSAAPGQKVTISCSGSSSNIGNNYV |
| | antibody | SWYQQLPGTAPKLLIYENNKRPSGISDRFSGSKS |
| | | GTSATLGITGLQTGDEADYYCATGDSGMTLVFGG |
| | | GTKLTVL |
| 65 | VH of anti-MOG MO3J-38 | MAGSLQVDQVQLVQSGPEVRKPGASVKVSCRAS |
| | antibody | GYTFTSNDINWVRQAAGQGLEYLGWLHPKSGGT |
| | | GYAQKFQGRVTMTRDTSISTAYLELSNLTSDDTA |
| | | VYYCARVSFDEVIDFWGQGTLVTVSS |
| 66 | VL of anti-MOG MO3J-38 | QSVLTQPPSASGTPGQRVTISCSGTRSNIGSNTV |
| | antibody | NWYQHLPGTAPKLLIYSNNQRPSGVPDRFSASKS |
| | | GTSASLAISGLQSEDEADYFCAAWDDSLNGVGFG |
| | | GGTKLTVL |
| 67 | VH of anti-MOG MO3J-40 | MAGSLQVDEVQLVESGAEVKKPGESLKISCKGSG |
| | antibody | YTFTSNWIGWVRQMPGKGLEWMGIIYPGDSDTR |
| | | YSPSFQGQGTISADKSISTAYLQWSSLRASDTAM |
| | | YYCARASIAVRPHIDYWGQGTLVTVSS |
| 68 | VL of anti-MOG MO3J-40 | DVVMTQSPLSLPVTLGQPASISCRSSQSLVYSDG |
| | antibody | NTYLNWFQQRPGQSPRRLIYKVSNRDSGVPDRF |
| | | SGSGSGTDFTLEISRVEAEDVGVYYCMQGTHWP |
| | | RTFGQGTKLEIK |
| 69 | VH of anti-MOG MO3J-43 | MAGSLQVDEVQLVESGGGLVKPGGSLRLSCAAS |
| | antibody | GFTFSDYYMSWIRQAPGKGLEWVSYISSSGNTIY |
| | | YADSVKGRFTISRDNAKNSLYLQMNSLRAEDTAV |
| | | YYCAKDSPVPTVWGQGTLVTVSS |
| 70 | VL of anti-MOG MO3J-43 | QSVVTQPPSVSGAPGQRVSISCTGGSSNIGADYD |
| | antibody | VHWYQQLPGTAPKLLIYGNNNRPSGVPDRFSGS |
| | | KSGTSASLAISGLQPEDEAVYYCQSYDSGLRSSV |
| | | FGGGTKLTVL |
| 71 | VH of anti-MOG MO3J-44 | MAGSLQVDQVQLVQSGAEVRKPGASVKISCQISG |
| | antibody | YNFISYTIQWVRQAPGQRPEWMGWINSGNGNTK |
| | | YSQKFQGRVTFTRDTSTSTAYMELSSLRSEDTAV |
| | | YYCARSGIGPWGQGTLVTVSS |
| 72 | VL of anti-MOG MO3J-44 | EIVLTQPPDLQSVTPKKKVTITCRASQSIGNSLHW |
| | antibody | CQQKPDQSPKLLIKYASQSISGVPSRFSGSGSGT |
| | | DFTLTINSLEAEDAATYYCHQSSSLPFTFGPGTKV |
| | | DIK |
| 73 | VH of anti-MOG MO3J-49 | MAGSLQVDQM.LVQSGAEVKKPGASVKVSCKAS |
| | antibody | GYTFTSYAMHWVRQAPGQRLEWMGWINAGNGN |
| | | TKYSQRFQGRVTITRDTSASTAYLELSSLRSEDTA |
| | | VYYCARAPLGLTANGGGFDPWGQGTLVTVSS |
| 74 | VL of anti-MOG MO3J-49 | DIVMTQSPSSLSASVGDRVTNTCRASQSISSYLS |
| | antibody | WYQQKPGKAPKLLIYIASSLQSGVPSRFSGTGSG |
| | | TDFTLTISSLQPEDFGTYYCQQSYSAPLTFGQGTK |
| | | VESK |
| 75 | VH of anti-MOG MO3J-51 | MAGSLQVDEVQLVQSGAEVKKPGASVKVSCKAS |
| | antibody | GYTFTSYGISWVRQAPGQGLEWMGWISAYNGNT |
| | | NYAQKLQGRVTMTTDTSTSTAYMELRSLGSDDTA |
| | | VYYCARNMGCSSTSCFVSWFDPWGQGTLVTVS |
| | | S |
| 76 | VL of anti-MOG MO3J-51 | QSVLTQPPSVSAAPGRKVTISCSGSSSNIGNNYV |
| | antibody | AWYQQLPGTAPKLLIYENNKRPSGIPGRFSGSKS |
| | | ATSATLGITGLQTGDEADYYCGTWDNSLSAWVF |
| | | GGGTKLTVL |
| 77 | VH of anti-MOG MO3J-52 | MAGSLQVDQMQLVQSGAEVKKPGSSVKVSCKAS |
| | antibody | GGTFSSYAISWVRQAPGQGLEWMGGIIPIFGTAN |
| | | YAQKFQGRVTITADESTSTAYMELSSLRSEDTAV |
| | | YYCARAREGLLVNYYGMDVWGQGTLVTVSS |
| 78 | VL of anti-MOG MO3J-52 | DIQMTQSPSTLSASVGDRVTTTCRASQGISNYLA |
| | antibody | WFQQKPGKAPKSLIYAASSLQSGVPSRFSGGGS |
| | | GTDFTLTINSLQPEDFATYYCLHDYNYPTFGQGTK |
| | | VEIK |
| 79 | VH of anti-MOG MO3I-56 | MAGSLQVDQVQLVESGGGVVQPGSSLRLSCTAS |
| | antibody | GFKFDDYAMHWVRQAPGKGLEWVSGISWNSGSI |
| | | GYADSVKGRFTISRDNAKNSLYLQMNSLRAEDTA |
| | | SYYCAKSLPHYYDSPPYGMDVWGQGTLVTVSS |
| 80 | VL of anti-MOG MO3I-56 | DIQLTQSPSSLSASVGDRVTITCRASQGISSALAW |
| | antibody | YQQKPGKAPKLLIYDASSLESGVPSRFSGSGSGT |
| | | DFTLTISSLQPEDFATYYCQHRGTFGGGTKVDIK |
| 81 | VH of anti-MOG MO3I-57 | MAGSLQVDQVQLVQSGAEVKKPGASVKVSCKAS |
| | antibody | GYTFSSYGISWVRQAPGQGLEWMGWISANTGNT |
| | | DYAERLQGRVTMTTDTSTTTAYMELRSLRSDDTA |
| | | VYYCARGAPNGYAVDYWGQGTLVTVSS |
| 82 | VL of anti-MOG MO3I-57 | QSVLTQPPSASGAPGQRVSISCSGSSSNIGTNHV |
| | antibody | YWYQQFTGMAPKLIIDTNNQRPPGVPARFSGSKS |
| | | GTSASLAISGLRSEDESDYYCLTWDDGLYDWVFG |
| | | GGTKLTVL |
| 83 | VH of anti-MOG MO3I-60 | MAGSLQVDEVQLVESGGGLVKPGGSLGLSCAAS |
| | antibody | GFTFTNAWGHWVRQAPGKGLEWVGRIKSKTDG |
| | | GTTDYAAPVKDRFSISRDDSKNTLYLQMNSPTTE |
| | | DTAVYYCATENGMDIVTTFDSWGQGTLVTVSS |
| 84 | VL of anti-MOG MO3I-60 | AIRMTQSPSSLSASVGDRVTITCRASQSIGSYLSW |
| | antibody | YRQKPGKAPKLLIYDSSTLQSGVASRFSGSGSGT |
| | | DFTLTISALQPEDFATYYCHQSYRTPLSFGGGTKV |
| | | EIK |
| 85 | VH of anti-MOG MO3I-63 | MAGSLQVDQVQLVQSGAEVKTPGASVKISCKAS |
| | antibody | GYAFTSYAMHWVRAPGQGLEWMGWINAANANT |
| | | KYSQRFQGRVTITRDTSASTAYMELNSLRSEDTA |
| | | VYYCASSEDISRSNYYNYYMDVWGKGTTVTVSS |
| 86 | VL of anti-MOG MO3I-63 | DIVMTQSPSSLSASVGDRVTITCRASQTITTSLAW |
| | antibody | FQHRPGKAPKLLIYSASSLQSGVPSRFSGSGSGT |
| | | DFTLTISSLQPEDFATYSCQQTYSAPPTFGGGTKV |
| | | EIK |
| 87 | VH of anti-MOG MO3I-69 | MAGSLQVDQVQLVQSGAEVKPGASVKVSCKASG |
| | antibody | YTFTYYYLHWVRQAPGQGLEWMGWINPNSGATI |
| | | FAQKFQGRVTLTRDTSISTAYLDLSRLRSDDTAVY |
| | | YCARASMAYQYHSDVDYWGLGTLVTVSS |
| 88 | VL of anti-MOG MO3I-69 | QSVLTQPPSVSGAPGQRVTISCTGSSSNIGAGYD |
| | antibody | VHWYQQLPGTAPKLLIYGNSNRPSGVPDRFSGS |
| | | KSGTSASLAITGLQAEDEADYYCQSYDSSLVVFG |
| | | GGTKLTVL |
| 89 | VH of anti-MOG MO4H- | MAGSLQVDQVQLVQSGAEVKKPGASVKVSCKAS |
| | 51 antibody | GYTFTSYGMHWVRQAPGQRLEWMGWINPGNGN |
| | | TKYSQKFQGRVTITRDTSASTAYMDLSSLRSEDT |
| | | AVYYCARLPRIGGWFDPWGQGTLVTVSS |
| 90 | VL of anti-MOG MO4H-51 | DIVMTQSPDSLAVSLGERTTIHCKSSQSVLYSSNN |
| | antibody | KDYLAWYQQKPGQPPKLLIYWASTRESGVPDRF |
| | | SGSGSGTDFTLTISSLQAEDVTVYYCHQYYSTPLT |
| | | FGQGTKLEIK |
| 91 | VH of anti-MOG MO4H- | MAGSLQVDQVQLQQSGPGLVKPSQTLSLTCAISG |
| | 55 antibody | DSVSSNSAAWNWIRQSPSRGLEWLGRTYYRSK |
| | | WYNDYAESVKSRMTVTSDTSKNQVSLHLNSVTP |
| | | EDTAVYYCAREHIAVPGVFDIWGQGTLVTVSS |
| 92 | VL of anti-MOG MO4H-55 | DVVMTQPPSASGTPGQGVTISCSGSSSNIGSNTV |
| | antibody | NWYQQLPGTAPKLLIYGSGQRPSGVPDRFSGSR |
| | | SGTSASLAISGLQSEDEADYYCAAWDDSLNGRVF |
| | | GQGTKVDIT |
| 93 | VH of the anti-MOG | MAGSLQVDEVQLVQPGAEVKKPGASVKVSCKAS |
| | MO4H-65 antibody | DYTFTSYGISWVRQAPGQGLEWMGWISAYNGNT |
| | | YYARKFGRVTMTTDTSTTTAYMELRRLRSEDTAV |
| | | YYCARSGVDNIDYLFDYWGQGTLVTVSS |
| 94 | VL of anti-MOG MO4H-65 | EIVMTQSPGTMSVSPGESATLSCRASQSVSSNLA |
| | antibody | WYQQKPGQAPRLLIYGASTRATGLPARFSGSGS |
| | | RTDFTLTISSLQPEDFATYYCQQTTSFPLTFSGGT |
| | | KLEIT |
| 95 | VH of anti-MOG MO4H- | MAGSLQVDQVQLVQSGAEVKKPGASVKVSCKAS |
| | 106 antibody | GYTFTTYNIHWMRQAPGQSLEWMGWISTGNGDT |
| | | EYSQKLQGSVTFTRDTSASTVYMDLNSLTPGDTA |
| | | VYSCARESLFVSSWYADYWGQGTLVTVSS |
| 96 | VL of anti-MOG MO4H- | DVVMTQSPLSLPVTLGQPASISCRSSQSLVYSDG |
| | 106 antibody | NTYLNWFQQRPGQSPRRLIYKVSDRDSGVPDRF |
| | | SGSGSGTDSTLKISRVEAEDVGVYYCMQGTHWP |
| | | YTLGQGTKLEIK |
| 97 | VH of anti-MOG MO4H- | MAGSLQVDEVQLVQSGAEVKKPGASVKVSCKVS |
| | 118 antibody | GYTLTELSMHWVRQAPGKGLEWMGGFDPEDGE |
| | | TIYAQKFQGRVTMTEDTSTDTAYMELSSLRSEDT |
| | | AVYYCATIGPKVAAHTYYFDYWGQGTLVTVSS |
| 98 | VL of anti-MOG MO4H- | DIQLTQSPSSLSASVGDRVTITCRASQTIVTYLNW |
| | 118 antibody | YQQKPGKAPNLLITDASSLQSGVPSRFSGTESGT |
| | | DFTLTISSLQPEDFGSYYCQSYMNPITFGQGTRLE |
| | | IN |
| 99 | VH of anti-MOG MO3J-72 | MAGSLQVDEVQLVESGGGSVKPGGSLRLSCAAS |
| | antibody | GFRFDDYAMHWVRQAPGKGLEWVSGISWNSGAI |
| | | GYADSVQGRFTISGDNAKNTLYLQMNGLRVEDTA |
| | | MYYCARDGHGDYPIDYWGQGTLVTVSS |
| 100 | VL of anti-MOG MO3J-72 | QSVLTQPPSVSGAPGQRVSISCTGSGSNIGAGFD |
| | antibody | VHWYQQVPGTTPKLLIYGNNNRPSGVPDRFSGS |
| | | TSATSASLAITGLQADDEADYYCQSYDRSLRYVF |
| | | GTGTKLTVL |
| 101 | VH of anti-MOG MO3J-81 | MAGSLQVDQVQLVQSGAEVKKPGSSVKVSCRAS |
| | antibody | GGTFTSYALGWVRQAPGQGL.WMEGIIPIFATPKY |
| | | AQNFQDRLTITADTSTRTAYMELSGLTSDDTAVYY |
| | | CASGIYIDFQDYYMDVWGNGTTVTVSS |
| 102 | VL of anti-MOG MO3J-81 | EIVLTQSPGTLSLSPGERATLSCRASESVSSSYLA |
| | antibody | WYQQKPGQAPRLLIYGASSRATGIPDRFSGSGSG |
| | | TDFTLTISRLEPEDFAVYYCQQHGSPPPWTFGQG |
| | | TKVEIK |
| 103 | VH of anti-MOG MO3I-30 | MAGSLQVDEVQLLESGGGLVQPGGSLRLSCVAS |
| | antibody | GFTFRSYWMHWVRQDPGEGLVWVSRVSGDGSS |
| | | TNYADSVKGRFVISRDNAKDTLYLQMYSLRGEDT |
| | | AVYYCLRGNDGYGNFDYWGQGTTVTVSS |
| 104 | VL of anti-MOG MO3I-30 | DVVMTQSPLSLPVTLGQPASISCRPSQSLVYSDG |
| | antibody | NTYLNWFQQRPGQSPRRLIYKVSNRDYVVPDRF |
| | | SGSGSGTDFTLKISRVEAEDVGVYYCMQGTHWP |
| | | LTFGGGTKVEIK |
| 105 | VH of anti-MOG MO3I-33 | MAGSLQVDQVQLVQSGAEVKPGATVKISCKVSG |
| | antibody | YTFTDYYMHWVQQAPGKGLEWMGLVDPEDGETI |
| | | YAEKFQGRVTITADTSTDTAYMELSSLRSEDTAVY |
| | | YCATSYHGTSGFDYWGQGTLVTVSS |
| 106 | VL of anti-MOG MO3I-33 | QSVVTQPPSVSGAPGQRVTIACTGSNSDIGAGHD |
| | antibody | VHWYQQFPRTAPKLIIFGNTNRPSGVPDRFSGSK |
| | | SGTSASLVITGLQADDEADYHCQSYDNNLSGPIF |
| | | GGGTKLTVL |
| 107 | VH of anti-MOG MO3I-34 | MAGSLQVDEVQLVQSGAEVKKPGASVKVSCKAS |
| | antibody | GYTFTSYGISWVRQAPGQGLEWMGWISAYNGNT |
| | | NYAQKLQGRVTMTTDTSTSTAYMELRSLRSDDTA |
| | | VYYCARNMGCSSTSCFVSWFDPWGQGTLVTVS |
| | | S |
| 108 | VL of anti-MOG MO3I-34 | QSVLTQPPSVSAAPGQKVTISCSGSRSNIGSNYV |
| | antibody | SWYQQLPGTAPKLLIYDNTRRPSGIPDRFYGSKS |
| | | GTSATLDITGLQTGDEADYHCATWDSSLSALLFG |
| | | GGTKVTVL |

SEQ ID NO: 27 = Nucleic sequence of the heavy chain of the recombinant
murine 8-18C5 antibody of sequence SEQ ID NO: 19:
GAAGTGAAGCTGCACGAGTCTGGCGCCGGACTGGTGAAACCTGGCGCCAGCGTGGAAA
TCAGCTGCAAGGCCACCGGCTACACCTTCAGCAGCTTTTGGATCGAGTGGGTGAAACAG
CGGCCTGGCCACGGCCTGGAATGGATCGGCGAGATCCTGCCCGGCAGAGGCCGGACC
AACTACAACGAGAAGTTCAAGGGCAAGGCCACATTCACCGCCGAGACAAGCAGCAACA
CCGCCTACATGCAGCTGAGCAGCCTGACCAGCGAGGACAGCGCCGTGTACTACTGCGC
CACCGGCAATACCATGGTGAACATGCCCTACTGGGGCCAGGGCACCACCGTGACCGTG
TCCAGCGCCAAGACCACCCCCCCCAGCGTGTACCCTCTGGCCCCTGGATCTGCCGCCC
AGACCAACAGCATGGTGACACTGGGCTGCCTGGTGAAAGGCTACTTCCCCGAGCCTGT
GACCGTGACCTGGAACAGCGGCTCCCTGAGCAGCGGCGTGCACACCTTCCCTGCCGTG
CTGGAAAGCGACCTGTACACCCTGTCCAGCAGCGTGACCGTGCCCTCCAGCCCCAGAC
CCAGCGAGACAGTGACCTGCAACGTGGCCCACCCCGCCAGCAGCACCAAGGTGGACAA
GAAAATCGTGCCCAGAGACTGCGGCTGCAAGCCCTGCATCTGCACCGTGCCCGAGGTG
TCCTCCGTGTTCATCTTCCCACCCAAGCCCAAGGACGTGCTGACCATCACCCTGACCCC
CAAAGTGACCTGCGTGGTGGTGGACATCAGCAAGGACGACCCCGAGGTGCAGTTCAGT
TGGTTCGTGGACGACGTGGAAGTGCACACCGCCCAGACACAGCCCAGAGAGGAACAGT
TCAACAGCACCTTCAGAAGCGTGTCCGAGCTGCCCATCATGCACCAGGACTGGCTGAAC
GGCAAAGAGTTCAAGTGCAGAGTGAACAGCGCCGCCTTCCCAGCCCCCATCGAGAAAA
CCATCAGCAAGACCAAGGGCAGACCCAAGGCCCCTCAGGTGTACACCATCCCCCCACC
CAAAGAACAGATGGCCAAGGACAAGGTGTCCCTGACCTGCATGATCACCGATTTCTTCC
CAGAGGACATCACCGTGGAATGGCAGTGGAACGGCCAGCCCGCCGAGAACTACAAGAA
CACCCAGCCCATCATGAACACCAACGGCAGCTACTTCGTGTACAGCAAGCTGAACGTGC
AGAAGTCCAACTGGGAGGCCGGCAACACCTTTACCTGCAGCGTGCTGCACGAGGGCCT
GCACAACCACCACACCGAGAAGTCCCTGAGCCACAGCCCCGGCAAG
SEQ ID NO: 28 = Nucleic sequence of the light chain of the recombinant
murine 8-18C5 antibody of sequence SEQ ID NO: 20:
GACATCGAGCTGACCCAGAGCCCTAGCAGCCTGGCCGTGTCTGCCGGCGAGAAAGTGA
CCATGAGCTGCAAGAGCAGCCAGAGCCTGCTGAACAGCGGCAACCAGAAGAACTACCT
GGCCTGGTATCAGCAGAAGCCCGGCCTGCCCCCCAAGCTGCTGATCTACGGCGCCAGC
ACCAGAGAAAGCGGCGTGCCCGACAGATTCACCGGCAGCGGCTCCGGCACCGACTTCA
CCCTGACCATCAGCAGCGTGCAGGCCGAGGATCTGGCCGTGTACTACTGCCAGAACGA
CCACAGCTACCCCCTGACCTTCGGAGCCGGCACCAAGCTGGAAATCAAGCGGGCCGAT
GCCGCCCCTACCGTGTCCATCTTCCCACCCAGCAGCGAGCAGCTGACCAGCGGCGGAG
CCAGCGTCGTGTGCTTCCTGAACAACTTCTACCCCAAGGACATCAACGTGAAGTGGAAG
ATCGACGGCAGCGAGCGGCAGAACGGCGTGCTGAACTCCTGGACCGACCAGGACAGC
AAGGACTCCACCTACAGCATGAGCAGCACCCTGACCCTGACCAAGGACGAGTACGAGC
GGCACAACAGCTACACATGCGAGGCCACCCACAAGACCAGCACCAGCCCCATCGTGAA
GTCCTTCAACCGGAACGAGTGC

An object of the present invention is also a method for obtaining an antibody according to the invention, comprising the following steps:
i) providing a nucleic acid sequence encoding an IgG heavy chain, said heavy chain comprising (a) in the variable domain, the 3 CDRs binding to an autoantigen, and (b) in the Fc fragment, a mutation of amino acid chosen from amino acids in position 240 to 243, 258 to 267 and 290 to 305, and preferably at least the E294del or Y300del mutation, the numbering being that of the EU index or equivalent in Kabat;
ii) a nucleic acid sequence encoding an IgG light chain is provided, said light chain comprising, in the variable domain, the 3 binding CDRs of the same autoantigen as that targeted in i); and
iii) the nucleic acid sequences obtained in i) and ii) are expressed in a host cell, and the antibody is recovered.

The nucleic acid sequence (polynucleotide or nucleotide sequence) encoding the IgG heavy chain comprises an Fc fragment having a mutation. The nucleic acid sequence encoding the heavy chain of IgG may be synthesized chemically (Young L and Dong Q., 2004, Nucleic Acids Res., April 1 5; 32 (7), Hoover, D M and Lubkowski, J. 2002, Nucleic Acids Res., 30, Villalobos A, et al., 2006. BMC Bioinformatics, June 6; 7: 285). The nucleotide sequence encoding the IgG heavy chain can also be amplified by PCR using suitable primers. The nucleotide sequence encoding the IgG heavy chain may also be cloned into an expression vector.

For example, the nucleic acid sequence SEQ ID NO: 27 (encoding the heavy chain SEQ ID NO: 19) may be used.

These techniques are described in detail in the reference manuals: Molecular cloning: a laboratory manual, 3rd edition-Sambrook and Russel eds. (2001) and Current Protocols in Molecular Biology—Ausubel et al. eds (2007).

The nucleic acid sequence provided in i) (polynucleotide), which encodes the parent polypeptide, is then modified to obtain a nucleic acid sequence encoding the variant.

This step is the actual mutation step. It may be carried out by any method known from the prior art, in particular by site-directed mutagenesis.

Preferably, the amino acid substitutions and deletions are carried out by site-directed mutagenesis, by the assembly PCR technique using oligonucleotides corresponding to the modifications inserted (see, for example, Zoller and Smith, 1982, Nucl. Acids Res. 10): 6487-6500; Kunkel, 1985, Proc. Natl. Acad. Sci USA 82: 488).

In step ii), a nucleic acid sequence encoding an IgG light chain is provided, said light chain comprising, in the variable domain, the 3 binding CDRs of the same autoantigen as that targeted in i).

For example, the nucleic acid sequence SEQ ID NO: 28 (encoding the light chain SEQ ID NO: 20) may be used.

Finally, in step iii), the nucleic acid sequences obtained in i) and ii) are expressed in a host cell, and the antibody thus obtained is recovered.

The nucleic acid sequences obtained in i) and ii) may be inserted into a bicistronic vector.

The cellular host may be chosen from prokaryotic or eukaryotic systems, for example bacterial cells but also yeast cells or animal cells, in particular mammalian cells. It is also possible to use insect cells or plant cells.

The preferred host cells are the rat line YB2/0, the hamster line CHO, in particular the CHO dhfr− and CHO Lec13 lines, the PER.C6™ line (Crucell), the HEK line in particular HEK293 (ATCC #CRL1573), the lines EB66, K562, NSO, SP2/0, BHK, HeLa, NIH/3T3 or COS. More preferably, the rat line YB2/0 is used. These host cells, for example CHO cells, can be transfected with at least one gene encoding a sialyltransferase.

Preferably, when the Fc fragment of the antibody according to the invention, in particular human, is modified relative to that of a parent antibody and consists of the Y300del mutation, it is produced in HEK cells such as HEK293 cells.

The polynucleotides encoding the heavy and light chains can also comprise codons optimized, in particular for its expression in certain cells (step iii)). The aim of codon optimization is to replace the natural codons with codons of which the transfer RNAs (tRNAs) carrying the amino acids are the most frequent in the cell type considered. The fact of mobilizing frequently encountered tRNAs has the major advantage of increasing the speed of translation of messenger RNAs (mRNAs) and therefore of increasing the final titer (Carton J M et al, Protein Expr Purif, 2007). Codon optimization also affects the prediction of secondary mRNA structures which could slow down reading by the ribosomal complex. Codon optimization also has an impact on the percentage of G/C which is directly related to the half-life of mRNAs and therefore to their translational potential (Chechetkin, J. of Theoretical Biology 242, 2006 922-934).

Codon optimization may be done by substitution of natural codons using codon frequency tables (codon Usage Table) for mammals and more particularly for Homo sapiens. There are algorithms available on the internet and made available by the suppliers of synthetic genes (DNA2.0, GeneArt, MWG, Genscript) which allow this sequence optimization to be carried out.

Preferably, the polynucleotides encoding the heavy and light chains comprise codons optimized for their expression in HEK cells, such as HEK293 cells, CHO cells, or YB2/0 cells. More preferably, the polynucleotides encoding the heavy and light chains comprise codons optimized for their expression in YB2/0 cells.

An object of the invention is also a composition comprising, in a physiologically acceptable medium, monoclonal antibodies according to the invention.

By “monoclonal antibody” or “monoclonal antibody composition”, or “mAb” for monoclonal Antibody, is meant a composition comprising antibody molecules having identical and unique antigenic specificity. The antibody molecules present in the composition are all encoded by the same heavy and light chain sequences and therefore have the same protein sequence.

An object of the invention is also the use of an antibody according to the invention, or the use of a composition as mentioned above, as a medicament.

The antibody according to the invention may be combined with pharmaceutically acceptable excipients, and optionally with sustained release matrices, such as biodegradable polymers, to form a therapeutic composition.

The pharmaceutical composition may be administered orally, sublingually, subcutaneously, intramuscularly, intravenously, intraarterially, intrathecally, intraocularly, intracerebrally, transdermally, pulmonary, locally or rectally. The active principle may then be administered in unit form of administration, in admixture with conventional pharmaceutical carriers. Unit administration forms include oral forms such as tablets, capsules, powders, granules and oral solutions or suspensions, sublingual and buccal administration forms, aerosols, subcutaneous implants, transdermal, topical, intraperitoneal, intramuscular, intravenous, subcutaneous, intrathecal, intranasal administration forms and rectal administration forms.

Preferably, the pharmaceutical composition contains a pharmaceutically acceptable vehicle for a formulation capable of being injected. They may in particular be isotonic, sterile formulas, saline solutions (with monosodium or disodium phosphate, sodium, potassium, calcium or magnesium chloride and the like, or mixtures of such salts), or lyophilized compositions, which, during the addition of sterilized water or physiological serum as appropriate, allow the constitution of injectable solutions.

Dosage forms suitable for injectable use include sterile aqueous solutions or dispersions, oily formulations including sesame oil, peanut oil, and sterile powders for the extemporaneous preparation of sterile injectable solutions or solutions. dispersions. In all cases, the form must be sterile and must be fluid insofar as it must be injected by syringe. It must be stable under the conditions of manufacture and storage and must be preserved against the contaminating action of microorganisms, such as bacteria and fungi.

The dispersions according to the invention can be prepared in glycerol, liquid polyethylene glycols or mixtures thereof, or in oils. Under normal conditions of storage and use, these preparations contain a preservative to prevent the growth of microorganisms.

The pharmaceutically acceptable carrier may be a solvent or dispersion medium containing, for example, water, ethanol, a polyol (e.g. glycerin, propylene glycol, polyethylene glycol, and the like), suitable mixtures of these, and/or vegetable oils. The proper fluidity may be maintained, for example, by the use of a surfactant, such as lecithin. The prevention of the action of microorganisms can be brought about by various antibacterial and antifungal agents, for example, parabens, chlorobutanol, phenol, sorbic acid or even thimerosal. In many cases, it will be preferable to include isotonic agents, for example, sugars or sodium chloride. Prolonged absorption of the injectable compositions can be brought about by the use in the compositions of agents delaying absorption, for example, aluminum monostearate or gelatin.

Sterile injectable solutions are prepared by incorporating the active substances in the required amount in the appropriate solvent along with several of the other ingredients listed above, as appropriate, followed by sterilization by filtration. Generally, dispersions are prepared by incorporating the sterilized active ingredients into a sterile vehicle which contains the basic dispersion medium and the required other ingredients from those listed above. In the case of sterile powders for the preparation of sterile injectable solutions, the preferred preparation methods are vacuum drying and lyophilization. In formulation, solutions will be administered in a manner compatible with the dosage formulation and in a therapeutically effective amount. The formulations are readily administered in a variety of dosage forms, such as the injectable solutions described above, but drug release capsules and the like can also be used. For parenteral administration in an aqueous solution, for example, the solution should be properly buffered and the liquid diluent made isotonic with sufficient saline or glucose. These particular aqueous solutions are particularly suitable for intravenous, intramuscular, subcutaneous and intraperitoneal administration. In this regard, sterile aqueous media which can be used are known to those skilled in the art. For example, a dose can be dissolved in 1 ml of isotonic NaCl solution and then added to 1000 ml of the appropriate liquid, or injected at the proposed site of the infusion. Certain variations in dosage may be applied depending on the condition of the subject being treated.

The pharmaceutical composition of the invention can be formulated in a therapeutic mixture comprising about 0.0001 to 1.0 milligrams, or about 0.001 to 0.1 milligrams, or about 0.1 to 1.0 milligrams, or even about 10 milligrams per dose or more. Multiple doses may also be administered. The specific therapeutically effective dose level for a particular patient may depend on a variety of factors, including the disorder being treated and the severity of the disease, the activity of the specific compound employed, the specific composition used, the age, the body weight, the general health, the sex and the diet of the patient, the time of administration, the route of administration, the rate of excretion of the specific compound used, the duration of the treatment, or the drugs used in parallel.

Preferably, the present invention relates to the use of an antibody according to the invention in the prevention and/or treatment of an autoimmune disease. It also relates to the use of a composition comprising monoclonal antibodies according to the invention for preventing and/or treating an autoimmune disease.

Preferably, the autoimmune disease is chosen from:
demyelinating diseases involving anti-MOG antibodies, such as multiple sclerosis;
Devic's neuromyelitis optic (NMO/NMOSD), in particular by targeting aquaporin-4 (AQP-4) and/or MOG;
type 1 diabetes, in particular by targeting the catalytic 2 subunit of glucose-6 phosphatase (IGRP). The IGRP is specific to the islets of Langerhans; and
rheumatoid arthritis, especially by targeting type 2 collagen.

Preferably, the antibody or composition according to the invention is used in the prevention and/or treatment of a demyelinating disease involving anti-MOG antibodies.

Such a disease is preferably selected from among acute disseminated encephalomyelitis (ADEM), Devic's neuromyelitis optic (NMO/NMOSD) and multiple sclerosis (MS).

Indeed, 40% of patients with acute disseminated encephalomyelitis (ADEM), which mainly occurs in children, are seropositive for anti-MOG antibodies. In Devic's neuromyelitis optic (NMO/NMOSD), a subgroup of adult anti-aquaporin-4 (AQP-4) seronegative patients show high titers of anti-MOG antibodies.

### FIGURES

FIG. 1: Pilot experiment of variants treated with antibody 8-18C5 in an EAE model with MOG₃₅₋₅₅

The mice were injected on day 7 with 50 μg of 8-18C5-Del antibody produced in YB2/0 cells ((“Del”, n=4), 8-18C5-WT produced in HEK cells ((“WT”, n=4), or an equivolume of PBS ((“PBS”, n=4). A) Clinical score, B) Kaplan Meier survival curve.

FIG. 2: Histograms of the absolute number of macrophages infiltrating the CD45^{hi} CD11b^{hi} CNS of mice treated with the 8-18C5-WT antibody produced in HEK cells (“WT”, n=3), the 8-18C5-Del variant produced in cells YB2/0 (“Del”) (n=2), or an equivolume of PBS ((“PBS”, n=4).

FIG. 3: Histograms of the absolute number of activated Foxp3+ regulatory T lymphocytes infiltrating the CNS on viable CD4+ Th1.2+ T cells of mice treated with the 8-18C5-WT antibody produced in HEK cells (“WT”, n=3), the 8-18C5-Del variant produced in YB2/0 (“Del”) cells (n=2), or an equivolume of PBS ((“PBS”, n=4). Data are plotted as mean+/−SEM.

FIG. 4: Western blot using a lectin (SNA) specific for a2.6 sialic acid for the different antibodies: 8-18C5-Del antibody produced in YB2/0 cells (“Del (YB2/0)”), antibody 8-18C5-WT produced in YB2/0 cells (“WT (YB2/0)”) and 8-18C5-WT antibody produced in HEK cells (“WT (HEK)”).

FIG. 5: Pilot experiment of variants treated with antibody 8-18C5 in a moderate model of EAE with MOG₃₅₋₅₅

The mice were injected on day 9 with 50 μg of antibody 8-18C5-Del (“Del”, n=8), 8-18C5-WT (“YB2”, n=8), or an equivalent of PBS ((“PBS”, n=7) A) Clinical score, B) Kaplan Meier survival curve.

### EXAMPLES

The following examples are given for the purpose of illustrating various embodiments of the invention.

### Example 1: Fc Cloning and Engineering of the Murine Monoclonal Antibody 8-18C5, and Characterization of the Bindings to Type I Fc Receptors, to FcRn and to its Antigen

Fc engineering was performed from a DNA vector encoding the recombinant murine mAb 8-18C5 clone of IgG1. The inventors created the in silico cloning construct by associating the sequences encoding a consensus constant domain with the variable domain (Fab) of mAb 8-18C5. The crystallized structure of the Fab 8-18C5 fragment is available in PDB (Protein Data Bank) under the accession number 1 PKQ. For the constant domain, the inventors have chosen a consensus sequence of a murine IgG1 (Mus musculus, IGHG1*01) listed in the online database IMGT (Immunogenetics). The sequences corresponding to the heavy and light chains have been synthesized in vitro and cloned into separate pCDNA3 vectors (e.g. Geneart). The two sequences were then subcloned into a single mammalian bicistronic vector, allowing the production of the murine mAb 8-18C5 (8-18C5-WT).

Then the inventors created a deletion homologous to the human E294Del deletion (the numbering being that of the EU index or equivalent in Kabat): they deleted the glutamic acid at position 171 of SEQ ID NO: 18 (constant region) of the Recombinant 8-18C5 mAb, in order to obtain the 8-18C5-Del variant.

Recombinant 8-18C5 murine antibody 8-18C5-WT was produced in HEK cells.

The murine 8-18C5 recombinant 8-18C5-WT and variant 8-18C5-Del antibodies were produced in YB2/0 cells to optimize the level of sialylation (50-90%). Advantageously, the YB2/0 cell line makes it possible to obtain an 8-18C5-Del variant, with a very high level of sialylation.

After production in YB2/0 cells (or also in HEK cells for 8-18C5-WT), the 8-18C5-WT and 8-18C5-Del antibodies were purified on protein G and characterized by SDS-PAGE and SEC, for validate their purity (>97%) and their integrity (aggregate rate <2%).

They were then characterized by ELISA on the FcRn and on the various FcγRs:

ELISA on FcRn (Human or Murine):

The binding of the 8-18C5-WT and 8-18C5-Del antibodies to FcRn was measured by a standard ELISA test. For this, Maxisorp immunoplates were coated with the recombinant human or murine FcRn proteins. After saturation of the plates with 5% PBS-LE, the solutions of 8-18C5-WT or 8-18C5-Del antibodies were added to each well at different concentrations (from 5 ng/mL to 0.5 μg/mL) and incubated for 1 h30 at 37° C. Goat anti-human (or anti-murine) IgG HRP F(ab′)2 were then incubated at 1/2500 for 1 hour 30 minutes at 37° C. The ELISA plates were then revealed with TMB (Pierce) and the absorbances were read at 450 nm.

ELISA on FcγRs (Human or Murine):

The binding of the 8-18C5-WT and 8-18C5-Del antibodies to human or murine FcγRs was measured by ELISA after incubation, with the F(ab′)2 of goat anti-human IgG HRP for 2 h at room temperature (at a final concentration of 0.5 μg/ml for each molecule) with gentle stirring. The IgGs aggregated to the F(ab′)2 were then incubated with gentle agitation for 1 h at 30° C. on the Maxisorp or NiNTA immunoplates previously coated with the FcγR and saturated with PBS-BSA 4%. The ELISA plates were then revealed with TMB (Pierce) and the absorbances were read at 450 nm.

ELISA characterization of the 8-18C5-WT and 8-18C5-Del antibodies confirmed that the introduction of a point deletion in the Fc domain did not affect the recognition of the antigen (using the recombinant MOG protein, rMOG). As shown in Table 1 below, it is striking that the binding to FcRn is not affected, while the binding to FcγRIII and FcγRIIB is reduced:

TABLE 1
Preliminary characterization of the 8-18C5-WT and
8-18C5-Del antibodies. Binding was evaluated by
ELISA on rMOG, and on type I and murine FcRs.

| | rMOG | FcγRI | FcγRIII | FcγRIV | FcγRIIB | FcRn |
|---|---|---|---|---|---|---|
| 8-18C5-WT | ++ | − | + | − | + | + |
| (product in | | | | | | |
| HEK ou | | | | | | |
| YB2/0) | | | | | | |
| 8-18C5-Del | ++ | − | − | − | − | + |

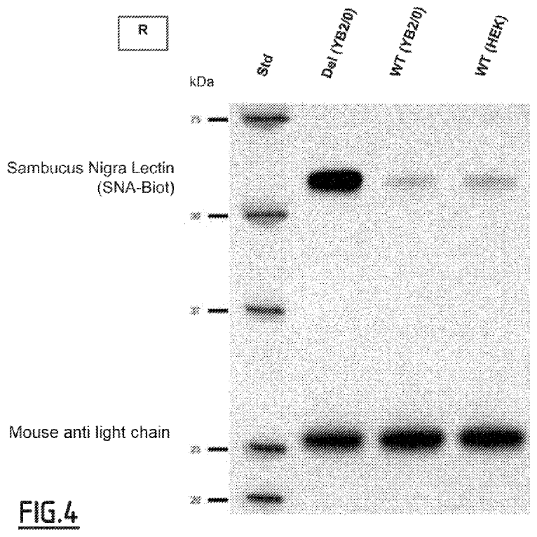

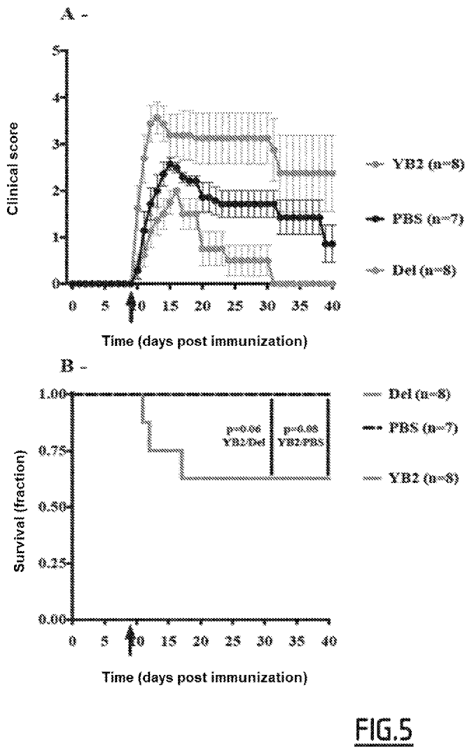

As the murine IgG1 isotype does not bind to FcγRIA (CD64) and FcγRIV, this indicates that the 8-18C5-Del variant no longer binds to any of the murine type I Fc receptors (FcRs). In addition, the increase in sialylation induced by the introduction of the “Del” mutation was confirmed by Western blot using a lectin (SNA) specific for a2.6 sialic acid (FIG. 4). For this, after the SDS-PAGE electrophoresis step, the antibodies were transferred onto a nitrocellulose membrane and then subjected to a Western Blot SNA: the conditions were as follows:

Saturation: TBS+BSA 1%+Tween-20 0.05%, overnight at 4° C.,

Washes: Phy Water+Tween-20 0.05%, 5×5 minutes, 1st incubation: Biotinylated SNA (VECTOR) at the 1/1000, 90 min at room temperature

2nd incubation: Streptavidin peroxidase at 1/2000, 60 min at room temperature

Chemiluminescence Detection

### Example 2: Impact of Fc Engineering Present in the mAb 8-18C5-Del Variant on Autoimmune Brain Disease

An intact blood-brain barrier prevents the infiltration of antibodies into the parenchyma of the CNS. It is therefore essential to induce mild autoimmune inflammation in the CNS to “prime” the tissue. This allows antibodies to enter the parenchyma and exercise their immune function.

The experimental model of choice is Experimental Autoimmune Encephalomyelitis (EAE), a crippling autoimmune inflammatory disease of the central nervous system. Since its description in 1933, it has served as a prototypical model of hypersensitivity (especially type IV) and a preclinical model of multiple sclerosis (MS) in which most of the currently marketed disease-modifying treatments have been validated. The most common form is active EAE, in which a demyelinating disease is induced by immunization with the linear 35-55 peptide of the MOG protein in C57Bl/6 mice (Ramadan A, Lucca L E, Carrie N, Desbois S, Axisa P P, Hayder M, Bauer J, Liblau R S, Mars L T. In situ expansion of T cells that recognize distinct self-antigens sustains autoimmunity in the CNS. Brain (2016) 139: 1433-1446). MAb 8-18C5 is specific for a conformational epitope of MOG (Breithaupt C, Schafer B, Pellkofer H, Huber R, Linington C, Jacob U. Demyelinating myelin oligodendrocyte glycoprotein-specific autoantibody response is focused on one dominant conformational epitope region in rodents. J Immunol (2008) 181: 1255-1263):

MAb 8-18C5 does not recognize the linear MOG35-55 peptide used for immunization, and only interacts with the intact native MOG protein present in the CNS. The immune-mediated effects induced by mAb 8-18C5 are therefore a consequence of binding to native MOG locally within inflammatory lesions.

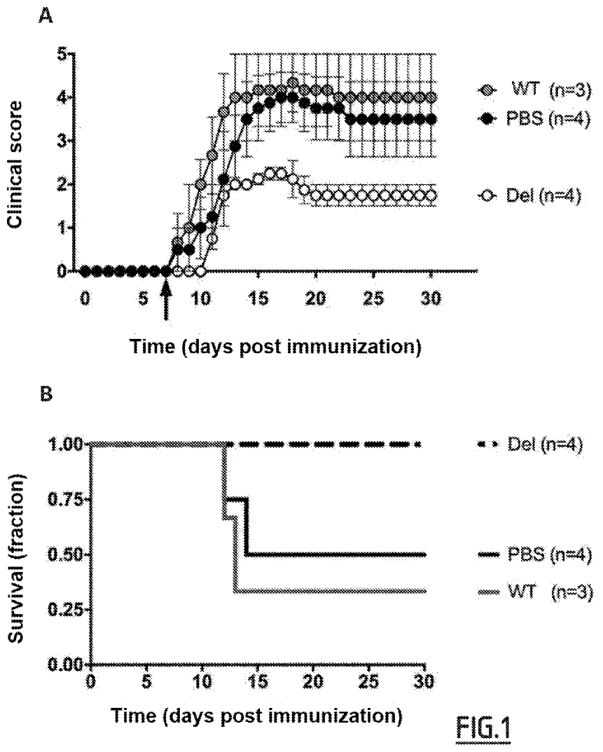

The impact of the 8-18C5-WT (produced in HEK) and 8-18C5-Del antibodies on experimental autoimmune encephalomyelitis (EAE) paralytic disease was determined in a pilot experiment, the results of which are shown in FIGS. 1 and 5.

Test 1:

The induction of EAE is carried out in C57Bl/6 mice by immunization with 50 μg of MOG35-55 in CFA (Complete Freund's adjuvant) comprising 600 μg of inactivated Mycobacterium tuberculosis H37RA, followed by 2 injections of pertussis toxin on day 0 (200 ng) and day 2 (400 ng).

7 days after immunization, each antibody was injected at a single dose of 50 μg/mouse. This 2.5 mg/kg dose of 8-18C5 antibodies is deliberately lower than the dose of IVIg to treat the same disease (4 g/kg in total: 4×1 g/kg).

The results are as follows:

Compared to control mice injected with PBS, mice treated with the 8-18C5-WT (WT) antibody showed worsened EAE, which resulted in the death of all of these mice 5-6 days after injection (FIG. 1B).

The 8-18C5-Del variant provides the opposite result: this variant not only lost its inherent pathogenicity (in this case the severity of the disease would have been similar to that of the mice treated with PBS), but it lessened the severity of disease. None of the mice treated with the 8-18C5-Del variant died, compared to 2 out of 4 mice for PBS (FIG. 1B), the severity of EAE stagnating at a clinical score of 2 which reflects a delay in clarification. The most severe stages of paralysis (>3) were never achieved (FIG. 1A).

Test 2:

Induction of moderate EAE is carried out in C57Bl/6 mice by immunization with 100 μg of MOG35-55 in CFA (Complete Freund's adjuvant) comprising 100 μg of inactivated Mycobacterium tuberculosis H37RA, followed by 2 injections of pertussis toxin to day 0 (200 ng) and day 2 (200 ng).

On day 9, two days before the induction of EAE on day 11, each antibody (murine 8-18C5 produced in YB2/0 (YB2) cells, variant carrying the E171 (Del) deletion (corresponding to the Del294 deletion in humans), is injected at a single dose of 50 μg/mouse PBS is injected into the control mice.

The results are as follows:

Compared to control mice injected with PBS, mice treated with the 8-18C5-WT antibody (YB2) showed worsened EAE, resulting in the death of 38% of the mice approximately 15 days after immunization (FIG. 5B and table 2) The 8-18C5-Del (Del) variant lost its inherent pathogenicity (in this case the severity of the disease would have been similar to that of the mice treated with PBS) and reduced the severity of the disease. The severity of EAE stagnates at a score of 2 and the most severe stages of paralysis are never achieved (FIG. 5A).

Finally, the 8-18C5-Del (Del) variant allows complete clinical recovery of all the mice, whereas the PBS treatment allows a clinical recovery of 57% of the mice and the treatment with the 8-18C5 antibody allows the clinical recovery of only 38% of animals (Table 2).

TABLE 2
Effects of treatments with PBS, 8-18C5WT 5YB2) and 8-18C5Del
(Del) on the mortality of mice and their clinical recovery.

| treatment IV | n | Mortality (%) | | Clinical recuperation (%) | |
|---|---|---|---|---|---|
| PBS | 7 | 0/7 | (0%) | 4/7 | (57%) |
| YB2 | 8 | 3/8 | (38%) | 3/8 | (38%) |
| Del | 8 | 0/8 | (0%) | 7/7 | (100%) |

### Conclusion:

The 8-18C5-Del variant improves EAE at a single dose, which is 400 times weaker than IVIG, and 40 times weaker than the recombinant sialylated variant F241A (described in Fiebiger B M, Maamary J, Pincetic A, Ravetch J V. Protection in antibody- and T cell-mediated autoimmune diseases by antiinflammatory IgG Fcs requires type II FcRs. Proc Natl Acad Sci USA (2015) 112: E2385-E2394). The critical difference between these parameters is that the 8-18C5 antibody recognizes an autoantigen linked to the disease.
The time of injection, i.e. just before the onset of the disease, strongly shows that the 8-18C5-Del variant has an effect on the ongoing pathological mechanisms. In addition, this effect is likely to occur locally in inflamed tissue, as the 8-18C5 antibody only recognizes native MOG protein, which is expressed exclusively in the central nervous system.

### Example 3: Effect of the 8-18C5-Del Variant on the Composition of the Cell Infiltrate in the Brain

Restoration of immune tolerance by low-dose autoantigen-induced mechanisms frequently results in the accumulation of FoxP3+ regulatory T cells. To determine whether the 8-18C5-Del variant can promote enrichment in FoxP3+ regulatory T cells, the inventors performed an experiment during which they evaluated the magnitude of the FoxP3+ regulatory T cell response in the brains of mice treated in Example 2.

On day 16 after immunization, the inventors isolated the mononuclear cells infiltrating the brain using a Percoll gradient, and analyzed the cellular composition of the immune infiltrate by flow cytometry.

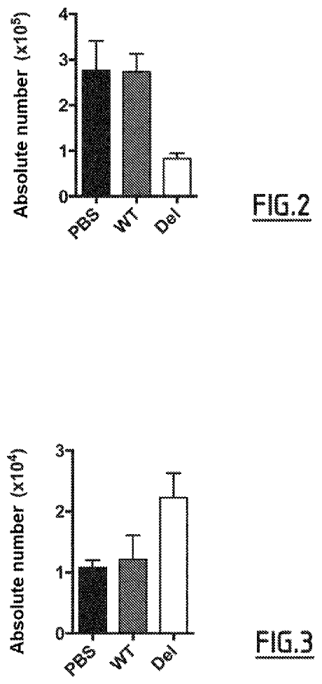

As shown in FIG. 2, the inflammatory activity in mice treated with the 8-18C5-Del variant is reduced, given the lower proportion and the total number of infiltrating macrophages compared to the PBS control group.

Concomitantly with the reduction in macrophages, CNS infiltration in mice treated with the 8-18C5-Del variant showed a notable increase in the proportion and absolute number of activated Foxp3+ regulatory T cells compared to EAE mice treated with PBS (FIG. 3).

### Conclusion:

These studies are consistent with a scenario in which the 8-18C5-Del variant re-educates the immune system by driving the expansion of regulatory T lymphocytes specific for the target MOG antigen. This mechanism likely requires that the MOG autoantigen be presented by tolerogenic antigen presenting cells (APCs), which preferentially activate regulatory T cells to the detriment of the pathogenic response of effector T cells.

This would identify the 8-18C5-Del variant according to the invention as a unique vector for selectively transferring autoantigen to tolerogenic APCs expressing type II Fc receptors.

### Example 4: Selection of Antibodies Equivalent to 8-18C5 by Phage Display

Selection of Human scFv Library (MG-UmAb):

During the selection steps, the human scFv library (MG-UmAb) was expressed on the surface of the bacteriophage M13 using standard procedures (Smith G P, Science 228: 1315 (1985)). E. coli XL1-Blue bacteria, containing the library to be expressed cloned in the vector pMG72, were cultured in 60 ml of 2YT medium supplemented with 100 μg/ml of ampicillin, 15 μg/ml of tetracycline and 1% (p/v) glucose at 30° C. The cells were then infected with the helper phage M13 (M13K07, Biolabs, bacteria/phage ratio=1/3) at 37° C. for 20 min and the production of phage-scFv was continued overnight at 26° C., at 230 rpm 2YT/ampicillin/glucose with 0.5 mM IPTG and 50 μg of kanamycin/ml. The next day, phages were precipitated with PEG6000 using standard protocols, resuspended in 1 ml of PBS buffer pH 7.4 and titrated infecting XL1-Blue cells.

For the solid phase selections, the phages-scFv diluted in PBS/4% skimmed milk/0.1% Tween 20 were incubated in 8 wells of Maxisorp plates (1-2×1011 phages/well in 100 μl final) coated beforehand. with the recombinant human MOG or Biotinylated MOG protein (on streptavidin plate) and blocked with 4% skimmed milk in PBS. After a 2 hour incubation at 37° C., the wells were washed 10 times with PBS/0.1% Tween 20 and twice with PBS. The selected phages were then eluted by infection with XL1-Blue bacteria in the exponential growth phase (2×150 μl/well, 20 min. At 37° C. without shaking). The infected bacteria were then plated on solid 2YT/ampicillin/glucose medium. The next day, cells were resuspended in 2YT medium with 15% glycerol, frozen and stored at −80° C. until the next round of selection.

For liquid phase selections, 4×10¹¹ phages were first incubated with biotinylated human MOG recombinant protein for 1 hour at room temperature with gentle shaking. Magnetic beads coated with streptavidin (Dynal) previously blocked with 4% skimmed milk in PBS were then added to the phages for 30 minutes at room temperature. The phage-bead complexes were washed 10 times with PBS/0.1% Tween 20 and 2 times with PBS using a magnet. The phage-bead complexes were then used to infect 5 ml of exponentially growing XL1-Blue bacteria, which were plated on solid 2YT/ampicillin/glucose medium. The next day, cells were resuspended in 2YT medium with 15% glycerol, frozen and stored at −80° C. until the next round of selection.

To ensure that specific scFvs were selected, several rounds of selection under different conditions (4-6 solid phase and/or 4-6 liquid phase) were implemented. The target concentrations (recombinant human MOG protein) were progressively reduced in order to select the most closely related scFvs. Selection rounds were also carried out on the homologous murine and cynomolgus MOG recombinant proteins in order to obtain scFvs which also recognize these proteins (similar screening conditions from the 2nd or 3rd round of selection). Finally, in order to obtain scFvs that target an epitope similar to the reference antibody 818-05, this antibody was used as a direct competitor in advanced selection steps (from the 4th round of selection).

The sequences of the scFvs obtained are as follows:

| SEQ ID NO: | Definition |
|---|---|
| 29 | VH of anti-MOG MO4H-03 antibody |
| 30 | VL of anti-MOG MO4H-03 antibody |
| 31 | VH of anti-MOG MO4H-04 antibody |
| 32 | VL of anti-MOG MO4H-04 antibody |
| 33 | VL of anti-MOG MO4H-37 antibody |
| 34 | VH anti-MOG MO4H-37 antibody |
| 35 | VH of anti-MOG MO4H-38 antibody |
| 36 | VL of anti-MOG MO4H-38 antibody |
| 37 | VH of anti-MOG MO4H-40 antibody |
| 38 | VL of anti-MOG MO4H-40 antibody |
| 39 | VH of anti-MOG MO4H-46 antibody |
| 40 | VL of anti-MOG MO4H-46 antibody |
| 41 | VH of anti-MOG MO3B-03 antibody |
| 42 | VL of anti-MOG MO3B-03 antibody |
| 43 | VH of anti-MOG MO3F-02 antibody |
| 44 | VL of anti-MOG MO3F-02 antibody |
| 45 | VH of anti-MOG MO4E-48 antibody |
| 46 | VL of anti-MOG MO4E-48 antibody |
| 47 | VH of anti-MOG MO4B-42 antibody |
| 48 | VL of anti-MOG MO4B-42 antibody |
| 49 | VH of anti-MOG MO4B-43 antibody |
| 50 | VL of anti-MOG MO4B-43 antibody |
| 51 | VH of anti-MOG MO3J-05 antibody |
| 52 | VL of anti-MOG MO3J-05 antibody |
| 53 | VH of anti-MOG MO3J-11 antibody |
| 54 | VL of anti-MOG MO3J-11 antibody |
| 55 | VH of anti-MOG MO3J-12 antibody |
| 56 | VL of anti-MOG MO3J-12 antibody |
| 57 | VH of anti-MOG MO3J-19 antibody |
| 58 | VL of anti-MOG MO3J-19 antibody |
| 59 | VH of anti-MOG MO3J-23 antibody |
| 60 | VL of anti-MOG MO3J-23 antibody |
| 61 | VH of anti-MOG MO3J-26 antibody |
| 62 | VL of anti-MOG MO3J-26 antibody |
| 63 | VH of anti-MOG MO3J-37 antibody |
| 64 | VL of anti-MOG MO3J-37 antibody |
| 65 | VH of anti-MOG MO3J-38 antibody |
| 66 | VL of anti-MOG MO3J-38 antibody |
| 67 | VH of anti-MOG MO3J-40 antibody |
| 68 | VL of anti-MOG MO3J-40 antibody |
| 69 | VH of anti-MOG MO3J-43 antibody |
| 70 | VL of anti-MOG MO3J-43 antibody |
| 71 | VH of anti-MOG MO3J-44 antibody |
| 72 | VL of anti-MOG MO3J-44 antibody |
| 73 | VH of anti-MOG MO3J-49 antibody |
| 74 | VL of anti-MOG MO3J-49 antibody |
| 75 | VH of anti-MOG MO3J-51 antibody |
| 76 | VL of anti-MOG MO3J-51 antibody |
| 77 | VH of anti-MOG MO3J-52 antibody |
| 78 | VL of anti-MOG MO3J-52 antibody |
| 79 | VH of anti-MOG MO3I-56 antibody |
| 80 | VL of anti-MOG MO3I-56 antibody |
| 81 | VH of anti-MOG MO3I-57 antibody |
| 82 | VL of anti-MOG MO3I-57 antibody |
| 83 | VH of anti-MOG MO3I-60 antibody |
| 84 | VL of anti-MOG MO3I-60 antibody |
| 85 | VH of anti-MOG MO3I-63 antibody |
| 86 | VL of anti-MOG MO3I-63 antibody |
| 87 | VH of anti-MOG MO3I-69 antibody |
| 88 | VL of anti-MOG MO3I-69 antibody |
| 89 | VH of anti-MOG MO4H-51 antibody |
| 90 | VL of anti-MOG MO4H-51 antibody |
| 91 | VH of anti-MOG MO4H-55 antibody |
| 92 | VL of anti-MOG MO4H-55 antibody |
| 93 | VH of anti-MOG MO4H-65 antibody |
| 94 | VL of anti-MOG MO4H-65 antibody |
| 95 | VH of anti-MOG MO4H-106 antibody |
| 96 | VL of anti-MOG MO4H-106 antibody |
| 97 | VH of anti-MOG MO4H-118 antibody |
| 98 | VL of anti-MOG MO4H-118 antibody |
| 99 | VH of anti-MOG MO3J-72 antibody |
| 100 | VL of anti-MOG MO3J-72 antibody |
| 101 | VH of anti-MOG MO3J-81 antibody |
| 102 | VL of anti-MOG MO3J-81 antibody |
| 103 | VH of anti-MOG MO3I-30 antibody |
| 104 | VL of anti-MOG MO3I-30 antibody |
| 105 | VH of anti-MOG MO3I-33 antibody |
| 106 | VL of anti-MOG MO3I-33 antibody |
| 107 | VH of anti-MOG MO3I-34 antibody |
| 108 | VL of anti-MOG MO3I-34 antibody |

Determination of the Binding to the MOG Protein: ELISA Tests of Phages-ScFv on the MOG Protein (Human and Murine):

The binding characteristics of the scFvs expressed on the surface of the phages isolated during the screening were determined using an ELISA assay using the recombinant MOG protein (R&D system). Along with the selected clones, phage-scFv-8-18C5 is expressed to serve as a positive control for ELISA. Briefly, phage-scFv were produced in the form of clones isolated on a 96-well plate in 800 μl of 2YT/ampicillin/glucose cultures infected with the helper phage M13K07 (as described previously). The phages produced overnight at 26° C. were then recovered in the supernatants after 30 minutes of centrifugation at 3000 g. These supernatants were directly diluted 1/2 in PBS/4% BSA/0.1% Tween 20 and tested on Maxisorp immunoplates previously coated with 0.5 μg of human or murine MOG or PBS (background control (bdf) in BSA)/well and blocked with 4% BSA in PBS. After incubation for 2 hours at 37° C., the wells were washed 3 times with PBS/0.1% Tween-20 and bound phages-scfv were detected with anti-M13 HRP antibody (GE Healthcare). The plate is read at 450 nm (OD) on a plate reader (TECAN). The results are expressed as a ratio: OD on target/OD bdf (OD on an uncoated plate saturated with BSA) and the ratios are compared with that obtained for the positive control.

### Results:

| | HMOG | MMOG |
|---|---|---|
| MO4H-03 | 5.14 | 9.12 |
| MO4H-04 | 9.36 | 9.78 |
| MO4H-37 | 4.24 | 4.75 |
| MO4H-38 | 4.36 | 6.04 |
| MO4H-40 | 4.19 | 4.72 |
| MO4H-46 | 6.57 | 4.68 |
| MO3B-03 | 6.23 | 5.55 |
| MO3F-02 | 13.77 | 22.13 |
| MO4E-48 | 10.07 | 7.78 |
| MO4B-42 | 6.11 | 6.68 |
| MO4B-43 | 5.82 | 9.56 |
| MO3J-05 | 16.41 | 7.53 |
| MO3J-11 | 16.08 | 12.43 |
| MO3J-12 | 16.36 | 8.99 |
| MO3J-19 | 11.72 | 5.66 |
| MO3J-23 | 15.95 | 7.46 |
| MO3J-26 | 14.59 | 3.62 |
| MO3J-37 | 12.87 | 6.66 |
| MO3J-38 | 18.36 | 5.59 |
| MO3J-40 | 20.99 | 6.48 |
| MO3J-43 | 20.84 | 8.26 |
| MO3J-44 | 14.51 | 6.61 |
| MO3J-49 | 42.63 | 33.32 |
| MO3J-51 | 14.58 | 4.76 |
| MO3J-52 | 16.29 | 6.86 |
| MO3I-56 | 2.29 | 2.29 |
| MO3I-57 | 2.44 | 2.77 |
| MO3I-60 | 2.09 | 2.75 |
| MO3I-63 | 2.66 | 5.55 |
| MO3I-69 | 2.26 | 2.76 |
| MO4H-51 | 3.46 | ND |
| MO4H-55 | 2.99 | ND |
| MO4H-65 | 2.44 | ND |
| MO4H-106 | 2.55 | 2.78 |
| MO4H-118 | 2.04 | 4.01 |
| MO3J-72 | 2.05 | 2.37 |
| MO3J-81 | 2.39 | 2.81 |
| MO3I-30 | 2.86 | 2.19 |
| MO3I-33 | 2.53 | 2.52 |
| MO3I-34 | 2.14 | 2.45 |

The generated clones described above have a higher ratio than the ratio of the 8-18C5 positive control; they therefore bind better to the MOG protein.

In addition, the following experiments may be carried out in addition:

### Determine the Impact on the T Cell Response:

On day 16 after immunization, the inventors isolated the mononuclear cells infiltrating the brain using a Percoll gradient, and analyzed the cellular composition of the immune infiltrate by flow cytometry. The amplitude of the responses of regulatory (Foxp3+) and pathogenic (Th1/Th17) T lymphocytes may be determined to formally show whether, in mice treated with 8-18C5-Del, the contraction of the pathogenic response is correlated with the expansion regulatory T cell response.

### Determine the Specificity of the Pathogenic and Immunoregulatory T Cell Response:

Using antigen booster experiments, MHC tetramers and T cells express a transgenic TCR specific for MOG35-55, the specificity of the regulatory and pathogenic T cell response can be established. The aim is to establish the central role of the MOG autoantigen in mediating the therapeutic effect of the monoclonal antibody 8-18C5.

Identify the Tolerogenic Myeloid Subset that Interacts with 8-18C5-Del:

During disease improvement, it is conceivable that the m8-18C5-Del variant (not bound to type I receptors) will bind to type II FcRs which include, inter alia, the CD209 receptor for the lectin of type C (SIGN-R1) and CD23. To identify the target cells of the 8-18C5-Del antibody, 2 approaches can be advantageously developed:
8-18C5-Del and 8-18C5-WT are labeled with distinct fluorochromes and the immune cells infiltrating the brain which bind either or both antibodies are analyzed by flow cytometry.
the profile of the type I or II Fc receptor on immune cells infiltrating the brain is established using a flow cytometry approach.

Particular attention is paid to DC-SIGN+ macrophages/immunoregulatory microglia and to anti-inflammatory M2 macrophages/microglia Arg-1+CD45+CD11 B+F4/80+CD68+. Myeloid-derived suppressor cells and plasmacytoid DCs are poorer candidates as they have been reported to aggravate EAE.

Like the strategies mentioned above, these approaches are performed on day 16 after immunization, by isolating the mononuclear cells infiltrating the brain using a Percoll gradient.

### Functional Implication of the Identified Tolerogenic Myeloid Subset:

After CNS isolation, the tolerogenic subsets are co-cultured with transgenic TCR T cells to demonstrate that presentation of the MOG antigen leads to expansion of regulatory T cells. Second, neutralizing antibody approaches are used in vivo to delay the induction or function of the cells involved.

1. Isotype G antibody directed against native myelin oligodendrocytic glycoprotein (MOG), comprising:
an Fc fragment exhibiting high sialylation, and
an Fab fragment capable of binding to the autoantigen.

2. Antibody according to claim 1, wherein the Fc fragment is modified relative to that of a parent antibody, and comprises at least one amino acid mutation chosen from amino acids in position 240 to 243, 258 to 267 and 290 to 305 of said Fc fragment, the numbering being that of the index EU or equivalent in Kabat.

3. Antibody according to claim 2, wherein the mutation is selected from V262del, V263F, V263K, V263W, V264K, V264P, D265A, D265E, D265G, D265L, D265S, D265V, V266A, V266P, V266S, V266T, S267N, S267P, S267R, S267W, P291C, P291V, P291Y, P291W, R292A, R292del, R292T, R292V, R292Y, E293del, E293F, E293P, E293W, E293Y, E294del, E294D, E294N, E294W, E294F, E293del/E294del, Q295D, Q295del, Q295F, Q295G, Q295K, Q295N, Q295R, Q295W, Y296A, Y296C, Y296del, Y296E, Y296G, Y296Q, Y296R, Y296V, S298del, S298E, S298F, S298G, S298L, S298M, S298N, S298P, S298R, S298T, S298W, S298Y, Y300D, Y300del, Y300G, Y300N, Y300P, Y300R, Y300S, R301A, R301F, R301G, R301H, R3011, R301K, R301Q, R301V, R301W, R301Y, V302del, V302A, V302F, V302G, V302P, V303A, V303C, V303P, V303L, V303S, V303Y, S304C, S304M, S304Q, S304T, V305F and V305L, the numbering being that of the index EU or equivalent in Kabat.

4. Antibody according to claim 1, wherein the Fc fragment is modified relative to that of a parent antibody and comprises at least the E294del mutation, the numbering being that of the EU index or equivalent in Kabat.

5. Antibody according to claim 1, characterized in that it is directed against native MOG and comprises the following 6 CDRs:
H-CDR1: SEQ ID NO: 11,
H-CDR2: SEQ ID NO: 12,
H-CDR3: SEQ ID NO: 13,
L-CDR1: SEQ ID NO: 14,
L-CDR2: GAS, and
L-CDR3: SEQ ID NO: 15.

6. Antibody according to claim 1, which is selected from human IgG1, IgG2, IgG3 and IgG4, preferably is an IgG1.

7. Antibody according to claim 1, which is chimeric, humanized or human.

8. Antibody according to claim 1, which comprises as heavy chain the sequence SEQ ID NO: 24, with the deletion of glutamic acid in position 294 in numbering of the index EU or equivalent in Kabat, and as light chain the sequence SEQ ID NO: 25.

9. Composition comprising, in a physiologically acceptable medium, monoclonal antibodies according to claim 1.

10-12. (canceled)

13. Method for treating a subject in need thereof, comprising administering to said subject an antibody according to claim 1, or a composition comprising the antibody and a physiologically acceptable medium.

14. Method for preventing and/or treating an autoimmune disease in a subject in need thereof, comprising administering to said subject an antibody according to claim 1, or a composition comprising the antibody and a physiologically acceptable medium.

15. Method for preventing and/or treating a demyelinating disease involving anti-MOG antibodies in a subject in need thereof, comprising administering to said subject an antibody according to claim 1, or composition comprising the antibody and a physiologically acceptable medium.

16. Method according to claim 12, wherein the demyelinating disease involving anti-MOG antibodies is chosen from acute disseminated encephalomyelitis, Devic's optic neuromyelitis and multiple sclerosis.


SEQUENCE LISTING (108 sequences)

SEQ ID NO: 1 - protein, 222 aa, Artificial Sequence
  Synthetic Human IgG1 Fc region G1m1 17 without upper hinge region
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 221 aa, Artificial Sequence
  Synthetic Human IgG2 Fc region G1m1 17 without upper hinge region
CPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 222 aa, Artificial Sequence
  Synthetic Human IgG3 Fc region without upper hinge region
CPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 4 - protein, 222 aa, Artificial Sequence
  Synthetic Human IgG4 Fc region without upper hinge region
CPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 222 aa, Artificial Sequence
  Synthetic Human IgG1 Fc region G1m3 without upper hinge region
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 232 aa, Artificial Sequence
  Synthetic Human IgG1 Fc region G1m1, 17 with upper hinge region
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 228 aa, Artificial Sequence
  Synthetic Human IgG2 Fc region with upper hinge region
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 8 - protein, 279 aa, Artificial Sequence
  Synthetic Human IgG3 Fc region with upper hinge region
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 9 - protein, 229 aa, Artificial Sequence
  Synthetic Human IgG4 Fc region with upper hinge region
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 232 aa, Artificial Sequence
  Synthetic Human IgG1 Fc region G1m3 with upper hinge region
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 8 aa, Artificial Sequence
  Synthetic Murine antibody 8-18C5 H-CDR1
GYTFSSFW

SEQ ID NO: 12 - protein, 8 aa, Artificial Sequence
  Synthetic Murine antibody 8-18C5 H-CDR2
ILPGRGRT

SEQ ID NO: 13 - protein, 11 aa, Artificial Sequence
  Synthetic H-CDR3 from murine antibody 8-18C5
ATGNTMVNMPY

SEQ ID NO: 14 - protein, 12 aa, Artificial Sequence
  Synthetic L-CDR1 of murine antibody 8-18C5
QSLLNSGNQKNY

SEQ ID NO: 15 - protein, 8 aa, Artificial Sequence
  Synthetic L-CDR3 of murine antibody 8-18C5
QNDHSYPL

SEQ ID NO: 16 - protein, 118 aa, Artificial Sequence
  Synthetic VH of the recombinant murine 8-18C5 antibody
EVKLHESGAGLVKPGASVEISCKATGYTFSSFWIEWVKQRPGHGLEWIGEILPGRGRTNYNEKFKGKATFTAETSSNTAYMQLSSLTSEDSAVYYCATGNTMVNMPYWGQGTTVTVSS

SEQ ID NO: 17 - protein, 113 aa, Artificial Sequence
  Synthetic VL of the recombinant murine 8-18C5 antibody
DIELTQSPSSLAVSAGEKVTMSCKSSQSLLNSGNQKNYLAWYQQKPGLPPKLLIYGASTRESGVPDRFTGSGSGTDFTLTISSVQAEDLAVYYCQNDHSYPLTFGAGTKLEIK

SEQ ID NO: 18 - protein, 324 aa, Artificial Sequence
  Synthetic Constant region (CH1-hinge-CH2-CH3) of recombinant murine antibody 8-18C5
AKTTPPSVYPLAPGSAAQTNSMVTLGCLVKGYFPEPVTVTWNSGSLSSGVHTFPAVLESDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 19 - protein, 442 aa, Artificial Sequence
  Synthetic Recombinant murine 8-18C5 heavy chain
EVKLHESGAGLVKPGASVEISCKATGYTFSSFWIEWVKQRPGHGLEWIGEILPGRGRTNYNEKFKGKATFTAETSSNTAYMQLSSLTSEDSAVYYCATGNTMVNMPYWGQGTTVTVSSAKTTPPSVYPLAPGSAAQTNSMVTLGCLVKGYFPEPVTVTWNSGSLSSGVHTFPAVLESDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 20 - protein, 220 aa, Artificial Sequence
  Synthetic Recombinant murine 8-18C5 antibody light chain
DIELTQSPSSLAVSAGEKVTMSCKSSQSLLNSGNQKNYLAWYQQKPGLPPKLLIYGASTRESGVPDRFTGSGSGTDFTLTISSVQAEDLAVYYCQNDHSYPLTFGAGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 21 - protein, 118 aa, Artificial Sequence
  Synthetic VH of murine hybridoma 8-18C5 antibody
QVQLQQSGAELMKPGASVEISCKATGYTFSSFWIEWVKQRPGHGLEWIGEILPGRGRTNYNEKFKGKATFTAETSSNTAYMQLSSLTSEDSAVYYCATGNTMVNMPYWGQGTTLTVSS

SEQ ID NO: 22 - protein, 113 aa, Artificial Sequence
  Synthetic VL of murine hybridoma 8-18C5 antibody
DIVMTQSPSSLSVSAGEKVTMSCKSSQSLLNSGNQKNYLAWYQQKPGLPPKLLIYGASTRESGVPDRFTGSGSGTDFTLTISSVQAEDLAVYYCQNDHSYPLTFGAGTKLELK

SEQ ID NO: 23 - protein, 324 aa, Artificial Sequence
  Synthetic Constant region (CH1-hinge-CH2-CH3) of murine hybridoma 8-18C5 antibody
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 24 - protein, 448 aa, Artificial Sequence
  Synthetic Heavy chain chimeric 8-18C5 antibody
EVKLHESGAGLVKPGASVEISCKATGYTFSSFWIEWVKQRPGHGLEWIGEILPGRGRTNYNEKFKGKATFTAETSSNTAYMQLSSLTSEDSAVYYCATGNTMVNMPYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 220 aa, Artificial Sequence
  Synthetic Light chain  chimeric 8-18C5 antibody
DIELTQSPSSLAVSAGEKVTMSCKSSQSLLNSGNQKNYLAWYQQKPGLPPKLLIYGASTRESGVPDRFTGSGSGTDFTLTISSVQAEDLAVYYCQNDHSYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 8 aa, Artificial Sequence
  Synthetic Native human MOG epitope
RDHSYQEE

SEQ ID NO: 27 - DNA, 1326 bp, Artificial Sequence
  Synthetic Nucleic sequence of the heavy chain of the recombinant murine 8-18C5 antibody of sequence SEQ ID NO 19
gaagtgaagctgcacgagtctggcgccggactggtgaaacctggcgccagcgtggaaatcagctgcaaggccaccggctacaccttcagcagcttttggatcgagtgggtgaaacagcggcctggccacggcctggaatggatcggcgagatcctgcccggcagaggccggaccaactacaacgagaagttcaagggcaaggccacattcaccgccgagacaagcagcaacaccgcctacatgcagctgagcagcctgaccagcgaggacagcgccgtgtactactgcgccaccggcaataccatggtgaacatgccctactggggccagggcaccaccgtgaccgtgtccagcgccaagaccaccccccccagcgtgtaccctctggcccctggatctgccgcccagaccaacagcatggtgacactgggctgcctggtgaaaggctacttccccgagcctgtgaccgtgacctggaacagcggctccctgagcagcggcgtgcacaccttccctgccgtgctggaaagcgacctgtacaccctgtccagcagcgtgaccgtgccctccagccccagacccagcgagacagtgacctgcaacgtggcccaccccgccagcagcaccaaggtggacaagaaaatcgtgcccagagactgcggctgcaagccctgcatctgcaccgtgcccgaggtgtcctccgtgttcatcttcccacccaagcccaaggacgtgctgaccatcaccctgacccccaaagtgacctgcgtggtggtggacatcagcaaggacgaccccgaggtgcagttcagttggttcgtggacgacgtggaagtgcacaccgcccagacacagcccagagaggaacagttcaacagcaccttcagaagcgtgtccgagctgcccatcatgcaccaggactggctgaacggcaaagagttcaagtgcagagtgaacagcgccgccttcccagcccccatcgagaaaaccatcagcaagaccaagggcagacccaaggcccctcaggtgtacaccatccccccacccaaagaacagatggccaaggacaaggtgtccctgacctgcatgatcaccgatttcttcccagaggacatcaccgtggaatggcagtggaacggccagcccgccgagaactacaagaacacccagcccatcatgaacaccaacggcagctacttcgtgtacagcaagctgaacgtgcagaagtccaactgggaggccggcaacacctttacctgcagcgtgctgcacgagggcctgcacaaccaccacaccgagaagtccctgagccacagccccggcaag

SEQ ID NO: 28 - DNA, 660 bp, Artificial Sequence
  Synthetic Nucleic sequence of the light chain of the recombinant murine 8-18C5 antibody of sequence SEQ ID NO 20
gacatcgagctgacccagagccctagcagcctggccgtgtctgccggcgagaaagtgaccatgagctgcaagagcagccagagcctgctgaacagcggcaaccagaagaactacctggcctggtatcagcagaagcccggcctgccccccaagctgctgatctacggcgccagcaccagagaaagcggcgtgcccgacagattcaccggcagcggctccggcaccgacttcaccctgaccatcagcagcgtgcaggccgaggatctggccgtgtactactgccagaacgaccacagctaccccctgaccttcggagccggcaccaagctggaaatcaagcgggccgatgccgcccctaccgtgtccatcttcccacccagcagcgagcagctgaccagcggcggagccagcgtcgtgtgcttcctgaacaacttctaccccaaggacatcaacgtgaagtggaagatcgacggcagcgagcggcagaacggcgtgctgaactcctggaccgaccaggacagcaaggactccacctacagcatgagcagcaccctgaccctgaccaaggacgagtacgagcggcacaacagctacacatgcgaggccacccacaagaccagcaccagccccatcgtgaagtccttcaaccggaacgagtgc

SEQ ID NO: 29 - protein, 125 aa, Artificial Sequence
  Synthetic VH
MAGSLQVDQVQLVQSGTEVKKPGASVKVSCKVSGYTLTELSMHWVRQAPGKGLEWMGGFDPEDGETIYAQKFQGRVTMTEDTSTDTAYMELSSLRSEDTAVYYCATGATGAFDIWGQGTTVTVSS

SEQ ID NO: 30 - protein, 112 aa, Artificial Sequence
  Synthetic VL
DIVMTQTPLSSPVTLGQPASISCRSSQSLVDSDGNTYLNWLQQRPGQPPRLLIYKISNRFSGVPDRFSGSGAGTEFTLKISRVEAEDVGVYYCMQATQFPHTFGQGTKLEIK

SEQ ID NO: 31 - protein, 133 aa, Artificial Sequence
  Synthetic VH
MAGSLQVDEVQLVQSGAEVKKPVASVKVSCKASGYTFTSYGISWVRQAPGQGLEWMGWISAYNGNTNYAQKLQGRVTMTTDTSTSTAYMELRSLRSDDTAVYYCARNMGCSSTSCFVSWFDPWGQGTLVTVSS

SEQ ID NO: 32 - protein, 110 aa, Artificial Sequence
  Synthetic VL
QSALTQPPSASGSPGQSVTISCTGTHSDVGSFDSVSWYQQHPDKAPKLIIYDVNKRPAGVPHRFSGSKSGNTASLTVSGLQSEDEADYYCNSYAGVDNFVFGTGTKVTVL

SEQ ID NO: 33 - protein, 127 aa, Artificial Sequence
  Synthetic VH
MAGSLQVDQMQLVSGAEVKKPGESLKISCKGSGYSFTSYWIGWVRQMPGKGLEWMGIIYPDDSDFRYSPSFQGRVTILLDRSINTAYLQLSSLQASDTAMYYCARREAVTAAPFDFWGQGTLVTVSS

SEQ ID NO: 34 - protein, 110 aa, Artificial Sequence
  Synthetic VL
QSVLTQPPSASGAPGQRVSISCSGSSSNIGTNHVYWYQQFTGMAPKLIIDTNNQRPSGVPDRFSGSKSGTSASLAISGLQSDDAADYYCAAWDDSLNGYGFGSGTQLTVL

SEQ ID NO: 35 - protein, 133 aa, Artificial Sequence
  Synthetic VH
MAGSLQVDEVQLLESGGGLVQPGGVPETLLCNLWIHLQLLDALGPPSSREGAGVGLTYNSDGSSTTYADSVKDRFTISRDNSKNTLYLQMNSLRADDTAVYYCAKEHRTGGDPGGLSWNFDLWGRGTLVTVSS

SEQ ID NO: 36 - protein, 111 aa, Artificial Sequence
  Synthetic VL
QSVLTQPASVSGSPGQSITISCTGTSRDVGRYNYVSWYQQHPGKAPKLMIYEGSKRPSGVPDRFSGSKSGNTASLSISGLQSEDEADYYCAAWDDTLNGEVFGTGTKVTVL

SEQ ID NO: 37 - protein, 124 aa, Artificial Sequence
  Synthetic VH
MAGSLQVDQVQLVESGGGLVQPGRSLRLSCAASGFTFDDYAMHWVRQAPGKGLEVSGISWNSGSIGYADSVKGRFTISRDNAKNSLYLQMNSLRGEDTAVYYCAKFPGGSIGYWGPGTLVTVSS

SEQ ID NO: 38 - protein, 104 aa, Artificial Sequence
  Synthetic VL
DIVMTQSPSTLSASVGDRVTITCRASQGIRNDLGWYQQKPGKAPKLLIYAASSLQSGVPSRFSGSGSGTDFTLTISSLQPEDFATYYCQQGRGTFGPGTKVEIK

SEQ ID NO: 39 - protein, 127 aa, Artificial Sequence
  Synthetic VH
MAGSLQVDQVQLVQSGAEVKKPGASVKVSCKASGYTFTSYAMHWVRQAPGQRLEWMGWINAGNGNAKYSQKFQGRVTLTRDTSASTAYMKLSSLRSEDTAVYYCARGAPTYRYFDLWGRGTLVTVSS

SEQ ID NO: 40 - protein, 110 aa, Artificial Sequence
  Synthetic VL
QSVLTQPPSVSAAPGQKVTISCSGSSSNIGNNYVAWYQQLPGTAPKLLIYDNNKRPSGIPDRFSGSKSGTSATLGITGLQTGDEADYYCGTSDSSLSAVVFGGGTKLTVL

SEQ ID NO: 41 - protein, 128 aa, Artificial Sequence
  Synthetic VH
MAGSLQVDQVQLQQSGPGLVRPSQTLSLTCAISGDSVSSSSAAWNWVRSPSRGLEWLGRTYYRSKWYYDYAVSVKNRIAINPDTSKNQFSLHLNSVTPEDTAVYYCATGWLRGHLDYWGQGTLVTVSS

SEQ ID NO: 42 - protein, 107 aa, Artificial Sequence
  Synthetic VL
AIQMTQSPSSVSASVGDRVTITCRATQSISTYLNWYQQKVGRGPKLLVYAASRLQTGVPSRFSGSGSGTDFTLTISSLQPEDSATYYCQQSYSAPPAFGGGTKVEIK

SEQ ID NO: 43 - protein, 128 aa, Artificial Sequence
  Synthetic VH
MAGSLQVDQVQLQQSGPGLVKPSQTLSLTCAISGDSVSSNSAAWNWIRKSPSRGLEWLGRTYYRSKWYNDYAVSVKSRITINPDTSKNQFSLQLSSVTPEDTAVYYCARASAGTFGYWGQGTLVTVSS

SEQ ID NO: 44 - protein, 108 aa, Artificial Sequence
  Synthetic VL
DIVMTQSPSSLSASVGDRVTMTCRASQTINTYLNWYQQKPGKAPKLLIYAASTLQSGVPSRFSGSGSGTDFSLTISSLQPEDFATYYCQHGYNNPPFTFGPGTKVDIK

SEQ ID NO: 45 - protein, 129 aa, Artificial Sequence
  Synthetic VH
MAGSLQVDQVQLQSGLGLVKPSQTLSLTCAISGDSVSSNSAAWNWIRQSPSRGLEWLGRTYYRSKWINDYAVSVKSRITINPDTSKNQFSLQLNSVTPEDTAVYYCARAGGGSGLLDPWGQGTLVTVSS

SEQ ID NO: 46 - protein, 107 aa, Artificial Sequence
  Synthetic VL
DVVMTQSPAVLSVTPGEKVTITCRASEGIGNYLYWYQQKPDQALKLLINYASQSISGVPSRFSGSGSGTDFTFSISSLEAEDAAVYFCLQSYRLPLTFGGGTKVEIK

SEQ ID NO: 47 - protein, 127 aa, Artificial Sequence
  Synthetic VH
MAGSLQVDQVQLVQSGAEVKRPGESLKISCEGSGYSFTSSWIGWVRQMPGKGLECMGIIYPGDSDTRYSPSFQGHVTISADKSISTAYLQWSSLRASDTAMYYCARAYHSDYGFDFWGQGTLVTVSS

SEQ ID NO: 48 - protein, 108 aa, Artificial Sequence
  Synthetic VL
EIVLTQPLSVSESPGKTVTISCTRSSGSIANNFVQWYQRRPGSSPTTVIYENDQRPSGVPDRFSGSIDSSSNSASLTITGLETQDEADYYCQSFNDDVGGGNSGGGTK

SEQ ID NO: 49 - protein, 128 aa, Artificial Sequence
  Synthetic VH
MAGSLQVDEVQLLESGGGLVPGGSLRLSCEVSGFSFSNHAMHWVRQAPGKALEHLSVLGSDGRSTYYADSVKGRFTISRDISKTTVYLQMGSLRPGDMGVYYCARGLYGDHWDASDLWGQGTMVTVSS

SEQ ID NO: 50 - protein, 107 aa, Artificial Sequence
  Synthetic VL
EIVMTQSPATLSVSPAERVILSCRASQSVGNNVAWFQQKPGQAPRLLIHGASSRATGIPTRFSGSGSGTELTLTISSLQSEDFAVYYCQQYGSAPITFGQGTRLEIK

SEQ ID NO: 51 - protein, 133 aa, Artificial Sequence
  Synthetic VH
MAGSLQVDEVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARNMGCSSTSCFVSWFDPWGQGTLVTVSS

SEQ ID NO: 52 - protein, 110 aa, Artificial Sequence
  Synthetic VL
QPVLTQPPSASGTPGQRVTISCPGSSSNIGSNTVNWYQQLPGTAPKLLIYSNNQRPSGVPDRFSGSKSGTSASLAISGLQSEDEADYYCAAWDDSLNGWVFGGGTKLTVL

SEQ ID NO: 53 - protein, 128 aa, Artificial Sequence
  Synthetic VH
MAGSLQVDQMQLVQSGAEVKKPGASVKVSCKAPGYTFTDYYIHWVRQAPGQGPEWMGWINPNSGGTNYAQKFGRVTMTRGTSISTAYMELSRLTSDDTAVYYCARDQRRSSPYYLGYWDQGTLVTVSS

SEQ ID NO: 54 - protein, 110 aa, Artificial Sequence
  Synthetic VL
QSVLTQPPSVSGAPGQRVTIPCTGSSSNIASYDVHWYQQLPGTAPKLLIYGNTNRPSGVPDRFSGSKSGTSASLAITGLQAEDEADYYCQSYDSSLSGSVFGGGTKLTVL

SEQ ID NO: 55 - protein, 132 aa, Artificial Sequence
  Synthetic VH
MAGSLQVDEVQLLESGGGLVQPGGSLRLSCAASGFTFSTYWMHWVRQAPGRGLVWVSRINTDGSSTDYADSVKGRFTISRDNAKNTLYLQMNSLRAEDTAVYSCARGGQLVAAANDNWLDPWGQGTLVTVSS

SEQ ID NO: 56 - protein, 107 aa, Artificial Sequence
  Synthetic VL
AIQLTQSPSSLSASAGDRVTITCRASQSINNYLNWYQQKPGKAPKVLIYGASNLQSGVPSRFSGSGSGTDFTLTISSLQPEDFATYYCQQSYSTPRTFGQGTKVEIK

SEQ ID NO: 57 - protein, 133 aa, Artificial Sequence; identical to SEQ ID NO: 51
  Synthetic VH

SEQ ID NO: 58 - protein, 110 aa, Artificial Sequence
  Synthetic VL
QSVLTQPPSASGTPGQRVTISCSGSTSNIGSQIVNWYQQLPGTAPRLIIYNDNERPSGVSDRFSGSKSDTSASLAISGLQSEDEADYYCAAWDDSLNGYVFGTGTKVTVL

SEQ ID NO: 59 - protein, 129 aa, Artificial Sequence
  Synthetic VH
MAGSLQVDQVQLVQSGAEVKPGASVKVSCKASGYTFTSYGISWVRQAPGQGLEWMGWISAYNGNTNYAQKLQGRVTMTTDTSTSTAYMELRSLRSDDTAVYYCARDPVGSLRPYYMDVWGEGTTVTVSS

SEQ ID NO: 60 - protein, 107 aa, Artificial Sequence
  Synthetic VL
DIVMTQSPSTLSASVGDRVTITCRASQSISTWLAWYQQKPGKAPKLLIYAASSLQSGVPSRFSGSGSGTDFTLTISSLQPEDFATYYCQQSYSTPRTFGQGTKVEIK

SEQ ID NO: 61 - protein, 120 aa, Artificial Sequence
  Synthetic VH
MAGSLQVDQVQLVQSGAEVKKPGSSVKVSCKASGGTFSTYTLSWVRQAPGKGLEWMGGFDPEDGETIYAQKFQGRVTMTEDTSTDTAYLELSSLRSDDTAVYYCAADEFWGPGTLVTVSS

SEQ ID NO: 62 - protein, 110 aa, Artificial Sequence
  Synthetic VL
QSVLTQPPSVSAAPGQTVTISCSGSSSNIGNNYVSWYQQLPGTAPKLLIYDNNKRPSGIPDRFSGSKSGTSATLGITGLQTGDEADYYCGTWDSSLSAVVFGGGTKLTVL

SEQ ID NO: 63 - protein, 131 aa, Artificial Sequence
  Synthetic VH
MAGSLQVDEVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDMRAVAGTEGAFDIWGQGTMVTVSS

SEQ ID NO: 64 - protein, 109 aa, Artificial Sequence
  Synthetic VL
QSVVTQPPSMSAAPGQKVTISCSGSSSNIGNNYVSWYQQLPGTAPKLLIYENNKRPSGISDRFSGSKSGTSATLGITGLQTGDEADYYCATGDSGMTLVFGGGTKLTVL

SEQ ID NO: 65 - protein, 126 aa, Artificial Sequence
  Synthetic VH
MAGSLQVDQVQLVQSGPEVRKPGASVKVSCRASGYTFTSNDINWVRQAAGQGLEYLGWLHPKSGGTGYAQKFQGRVTMTRDTSISTAYLELSNLTSDDTAVYYCARVSFDEVIDFWGQGTLVTVSS

SEQ ID NO: 66 - protein, 110 aa, Artificial Sequence
  Synthetic VL
QSVLTQPPSASGTPGQRVTISCSGTRSNIGSNTVNWYQHLPGTAPKLLIYSNNQRPSGVPDRFSASKSGTSASLAISGLQSEDEADYFCAAWDDSLNGVGFGGGTKLTVL

SEQ ID NO: 67 - protein, 128 aa, Artificial Sequence
  Synthetic VH
MAGSLQVDEVQLVESGAEVKKPGESLKISCKGSGYTFTSNWIGWVRQMPGKGLEWMGIIYPGDSDTRYSPSFQGQGTISADKSISTAYLQWSSLRASDTAMYYCARASIAVRPHIDYWGQGTLVTVSS

SEQ ID NO: 68 - protein, 112 aa, Artificial Sequence
  Synthetic VL
DVVMTQSPLSLPVTLGQPASISCRSSQSLVYSDGNTYLNWFQQRPGQSPRRLIYKVSNRDSGVPDRFSGSGSGTDFTLEISRVEAEDVGVYYCMQGTHWPRTFGQGTKLEIK

SEQ ID NO: 69 - protein, 124 aa, Artificial Sequence
  Synthetic VH
MAGSLQVDEVQLVESGGGLVKPGGSLRLSCAASGFTFSDYYMSWIRQAPGKGLEWVSYISSSGNTIYYADSVKGRFTISRDNAKNSLYLQMNSLRAEDTAVYYCAKDSPVPTVWGQGTLVTVSS

SEQ ID NO: 70 - protein, 111 aa, Artificial Sequence
  Synthetic VL
QSVVTQPPSVSGAPGQRVSISCTGGSSNIGADYDVHWYQQLPGTAPKLLIYGNNNRPSGVPDRFSGSKSGTSASLAISGLQPEDEAVYYCQSYDSGLRSSVFGGGTKLTVL

SEQ ID NO: 71 - protein, 122 aa, Artificial Sequence
  Synthetic VH
MAGSLQVDQVQLVQSGAEVRKPGASVKISCQISGYNFISYTIQWVRQAPGQRPEWMGWINSGNGNTKYSQKFQGRVTFTRDTSTSTAYMELSSLRSEDTAVYYCARSGIGPWGQGTLVTVSS

SEQ ID NO: 72 - protein, 107 aa, Artificial Sequence
  Synthetic VL
EIVLTQPPDLQSVTPKKKVTITCRASQSIGNSLHWCQQKPDQSPKLLIKYASQSISGVPSRFSGSGSGTDFTLTINSLEAEDAATYYCHQSSSLPFTFGPGTKVDIK

SEQ ID NO: 73 - protein, 130 aa, Artificial Sequence
  Synthetic VH
MAGSLQVDQMLVQSGAEVKKPGASVKVSCKASGYTFTSYAMHWVRQAPGQRLEWMGWINAGNGNTKYSQRFQGRVTITRDTSASTAYLELSSLRSEDTAVYYCARAPLGLTANGGGFDPWGQGTLVTVSS

SEQ ID NO: 74 - protein, 107 aa, Artificial Sequence
  Synthetic VL
DIVMTQSPSSLSASVGDRVTNTCRASQSISSYLSWYQQKPGKAPKLLIYIASSLQSGVPSRFSGTGSGTDFTLTISSLQPEDFGTYYCQQSYSAPLTFGQGTKVESK

SEQ ID NO: 75 - protein, 133 aa, Artificial Sequence
  Synthetic VH
MAGSLQVDEVQLVQSGAEVKKPGASVKVSCKASGYTFTSYGISWVRQAPGQGLEWMGWISAYNGNTNYAQKLQGRVTMTTDTSTSTAYMELRSLGSDDTAVYYCARNMGCSSTSCFVSWFDPWGQGTLVTVSS

SEQ ID NO: 76 - protein, 110 aa, Artificial Sequence
  Synthetic VL
QSVLTQPPSVSAAPGRKVTISCSGSSSNIGNNYVAWYQQLPGTAPKLLIYENNKRPSGIPGRFSGSKSATSATLGITGLQTGDEADYYCGTWDNSLSAWVFGGGTKLTVL

SEQ ID NO: 77 - protein, 131 aa, Artificial Sequence
  Synthetic VH
MAGSLQVDQMQLVQSGAEVKKPGSSVKVSCKASGGTFSSYAISWVRQAPGQGLEWMGGIIPIFGTANYAQKFQGRVTITADESTSTAYMELSSLRSEDTAVYYCARAREGLLVNYYGMDVWGQGTLVTVSS

SEQ ID NO: 78 - protein, 106 aa, Artificial Sequence
  Synthetic VL
DIQMTQSPSTLSASVGDRVTTTCRASQGISNYLAWFQQKPGKAPKSLIYAASSLQSGVPSRFSGGGSGTDFTLTINSLQPEDFATYYCLHDYNYPTFGQGTKVEIK

SEQ ID NO: 79 - protein, 132 aa, Artificial Sequence
  Synthetic VH
MAGSLQVDQVQLVESGGGVVQPGSSLRLSCTASGFKFDDYAMHWVRQAPGKGLEWVSGISWNSGSIGYADSVKGRFTISRDNAKNSLYLQMNSLRAEDTASYYCAKSLPHYYDSPPYGMDVWGQGTLVTVSS

SEQ ID NO: 80 - protein, 103 aa, Artificial Sequence
  Synthetic VL
DIQLTQSPSSLSASVGDRVTITCRASQGISSALAWYQQKPGKAPKLLIYDASSLESGVPSRFSGSGSGTDFTLTISSLQPEDFATYYCQHRGTFGGGTKVDIK

SEQ ID NO: 81 - protein, 127 aa, Artificial Sequence
  Synthetic VH
MAGSLQVDQVQLVQSGAEVKKPGASVKVSCKASGYTFSSYGISWVRQAPGQGLEWMGWISANTGNTDYAERLQGRVTMTTDTSTTTAYMELRSLRSDDTAVYYCARGAPNGYAVDYWGQGTLVTVSS

SEQ ID NO: 82 - protein, 110 aa, Artificial Sequence
  Synthetic VL
QSVLTQPPSASGAPGQRVSISCSGSSSNIGTNHVYWYQQFTGMAPKLIIDTNNQRPPGVPARFSGSKSGTSASLAISGLRSEDESDYYCLTWDDGLYDWVFGGGTKLTVL

SEQ ID NO: 83 - protein, 131 aa, Artificial Sequence
  Synthetic VH
MAGSLQVDEVQLVESGGGLVKPGGSLGLSCAASGFTFTNAWGHWVRQAPGKGLEWVGRIKSKTDGGTTDYAAPVKDRFSISRDDSKNTLYLQMNSPTTEDTAVYYCATENGMDIVTTFDSWGQGTLVTVSS

SEQ ID NO: 84 - protein, 107 aa, Artificial Sequence
  Synthetic VL
AIRMTQSPSSLSASVGDRVTITCRASQSIGSYLSWYRQKPGKAPKLLIYDSSTLQSGVASRFSGSGSGTDFTLTISALQPEDFATYYCHQSYRTPLSFGGGTKVEIK

SEQ ID NO: 85 - protein, 132 aa, Artificial Sequence
  Synthetic VH
MAGSLQVDQVQLVQSGAEVKTPGASVKISCKASGYAFTSYAMHWVRAPGQGLEWMGWINAANANTKYSQRFQGRVTITRDTSASTAYMELNSLRSEDTAVYYCASSEDISRSNYYNYYMDVWGKGTTVTVSS

SEQ ID NO: 86 - protein, 107 aa, Artificial Sequence
  Synthetic VL
DIVMTQSPSSLSASVGDRVTITCRASQTITTSLAWFQHRPGKAPKLLIYSASSLQSGVPSRFSGSGSGTDFTLTISSLQPEDFATYSCQQTYSAPPTFGGGTKVEIK

SEQ ID NO: 87 - protein, 129 aa, Artificial Sequence
  Synthetic VH
MAGSLQVDQVQLVQSGAEVKPGASVKVSCKASGYTFTYYYLHWVRQAPGQGLEWMGWINPNSGATIFAQKFQGRVTLTRDTSISTAYLDLSRLRSDDTAVYYCARASMAYQYHSDVDYWGLGTLVTVSS

SEQ ID NO: 88 - protein, 109 aa, Artificial Sequence
  Synthetic VL
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLVVFGGGTKLTVL

SEQ ID NO: 89 - protein, 127 aa, Artificial Sequence
  Synthetic VH
MAGSLQVDQVQLVQSGAEVKKPGASVKVSCKASGYTFTSYGMHWVRQAPGQRLEWMGWINPGNGNTKYSQKFQGRVTITRDTSASTAYMDLSSLRSEDTAVYYCARLPRIGGWFDPWGQGTLVTVSS

SEQ ID NO: 90 - protein, 113 aa, Artificial Sequence
  Synthetic VL
DIVMTQSPDSLAVSLGERTTIHCKSSQSVLYSSNNKDYLAWYQQKPGQPPKLLIYWASTRESGVPDRFSGSGSGTDFTLTISSLQAEDVTVYYCHQYYSTPLTFGQGTKLEIK

SEQ ID NO: 91 - protein, 131 aa, Artificial Sequence
  Synthetic VH
MAGSLQVDQVQLQQSGPGLVKPSQTLSLTCAISGDSVSSNSAAWNWIRQSPSRGLEWLGRTYYRSKWYNDYAESVKSRMTVTSDTSKNQVSLHLNSVTPEDTAVYYCAREHIAVPGVFDIWGQGTLVTVSS

SEQ ID NO: 92 - protein, 110 aa, Artificial Sequence
  Synthetic VL
DVVMTQPPSASGTPGQGVTISCSGSSSNIGSNTVNWYQQLPGTAPKLLIYGSGQRPSGVPDRFSGSRSGTSASLAISGLQSEDEADYYCAAWDDSLNGRVFGQGTKVDIT

SEQ ID NO: 93 - protein, 128 aa, Artificial Sequence
  Synthetic VH
MAGSLQVDEVQLVQPGAEVKKPGASVKVSCKASDYTFTSYGISWVRQAPGQGLEWMGWISAYNGNTYYARKFGRVTMTTDTSTTTAYMELRRLRSEDTAVYYCARSGVDNIDYLFDYWGQGTLVTVSS

SEQ ID NO: 94 - protein, 107 aa, Artificial Sequence
  Synthetic VL
EIVMTQSPGTMSVSPGESATLSCRASQSVSSNLAWYQQKPGQAPRLLIYGASTRATGLPARFSGSGSRTDFTLTISSLQPEDFATYYCQQTTSFPLTFSGGTKLEIT

SEQ ID NO: 95 - protein, 129 aa, Artificial Sequence
  Synthetic VH
MAGSLQVDQVQLVQSGAEVKKPGASVKVSCKASGYTFTTYNIHWMRQAPGQSLEWMGWISTGNGDTEYSQKLQGSVTFTRDTSASTVYMDLNSLTPGDTAVYSCARESLFVSSWYADYWGQGTLVTVSS

SEQ ID NO: 96 - protein, 112 aa, Artificial Sequence
  Synthetic VL
DVVMTQSPLSLPVTLGQPASISCRSSQSLVYSDGNTYLNWFQQRPGQSPRRLIYKVSDRDSGVPDRFSGSGSGTDSTLKISRVEAEDVGVYYCMQGTHWPYTLGQGTKLEIK

SEQ ID NO: 97 - protein, 131 aa, Artificial Sequence
  Synthetic VH
MAGSLQVDEVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATIGPKVAAHTYYFDYWGQGTLVTVSS

SEQ ID NO: 98 - protein, 106 aa, Artificial Sequence
  Synthetic VL
DIQLTQSPSSLSASVGDRVTITCRASQTIVTYLNWYQQKPGKAPNLLITDASSLQSGVPSRFSGTESGTDFTLTISSLQPEDFGSYYCQSYMNPITFGQGTRLEIN

SEQ ID NO: 99 - protein, 127 aa, Artificial Sequence
  Synthetic VH
MAGSLQVDEVQLVESGGGSVKPGGSLRLSCAASGFRFDDYAMHWVRQAPGKGLEWVSGISWNSGAIGYADSVQGRFTISGDNAKNTLYLQMNGLRVEDTAMYYCARDGHGDYPIDYWGQGTLVTVSS

SEQ ID NO: 100 - protein, 110 aa, Artificial Sequence
  Synthetic VL
QSVLTQPPSVSGAPGQRVSISCTGSGSNIGAGFDVHWYQQVPGTTPKLLIYGNNNRPSGVPDRFSGSTSATSASLAITGLQADDEADYYCQSYDRSLRYVFGTGTKLTVL

SEQ ID NO: 101 - protein, 129 aa, Artificial Sequence
  Synthetic VH
MAGSLQVDQVQLVQSGAEVKKPGSSVKVSCRASGGTFTSYALGWVRQAPGQGLWMEGIIPIFATPKYAQNFQDRLTITADTSTRTAYMELSGLTSDDTAVYYCASGIYIDFQDYYMDVWGNGTTVTVSS

SEQ ID NO: 102 - protein, 109 aa, Artificial Sequence
  Synthetic VL
EIVLTQSPGTLSLSPGERATLSCRASESVSSSYLAWYQQKPGQAPRLLIYGASSRATGIPDRFSGSGSGTDFTLTISRLEPEDFAVYYCQQHGSPPPWTFGQGTKVEIK

SEQ ID NO: 103 - protein, 127 aa, Artificial Sequence
  Synthetic VH
MAGSLQVDEVQLLESGGGLVQPGGSLRLSCVASGFTFRSYWMHWVRQDPGEGLVWVSRVSGDGSSTNYADSVKGRFVISRDNAKDTLYLQMYSLRGEDTAVYYCLRGNDGYGNFDYWGQGTTVTVSS

SEQ ID NO: 104 - protein, 112 aa, Artificial Sequence
  Synthetic VL
DVVMTQSPLSLPVTLGQPASISCRPSQSLVYSDGNTYLNWFQQRPGQSPRRLIYKVSNRDYVVPDRFSGSGSGTDFTLKISRVEAEDVGVYYCMQGTHWPLTFGGGTKVEIK

SEQ ID NO: 105 - protein, 126 aa, Artificial Sequence
  Synthetic VH
MAGSLQVDQVQLVQSGAEVKPGATVKISCKVSGYTFTDYYMHWVQQAPGKGLEWMGLVDPEDGETIYAEKFQGRVTITADTSTDTAYMELSSLRSEDTAVYYCATSYHGTSGFDYWGQGTLVTVSS

SEQ ID NO: 106 - protein, 111 aa, Artificial Sequence
  Synthetic VL
QSVVTQPPSVSGAPGQRVTIACTGSNSDIGAGHDVHWYQQFPRTAPKLIIFGNTNRPSGVPDRFSGSKSGTSASLVITGLQADDEADYHCQSYDNNLSGPIFGGGTKLTVL

SEQ ID NO: 107 - protein, 133 aa, Artificial Sequence; identical to SEQ ID NO: 51
  Synthetic VH

SEQ ID NO: 108 - protein, 110 aa, Artificial Sequence
  Synthetic VL
QSVLTQPPSVSAAPGQKVTISCSGSRSNIGSNYVSWYQQLPGTAPKLLIYDNTRRPSGIPDRFYGSKSGTSATLDITGLQTGDEADYHCATWDSSLSALLFGGGTKVTVL